(19) United States
(12) Patent Application Publication
(10) Pub. No.: US 2026/0279489 A1
(43) Pub. Date: Sep. 17, 2026

(54) GENERATIVE MACHINE LEARNING MODELS FOR OMICS DATA AND PHENOTYPE DATA

(71) Applicant: NEUMORA THERAPEUTICS, INC., San Francisco, CA (US)

(72) Inventors: Tathagata Banerjee, Waltham, MA (US); John Van Wicheren Reynders III, Newton, MA (US)

(21) Appl. No.: 19/471,623

(22) PCT Filed: Apr. 5, 2024
(86) PCT No.: PCT/US2024/023281

§ 371 Date: Oct. 1, 2025

Related U.S. Application Data
(60) Provisional application No. 63/458,564, filed on Apr. 11, 2023.
(60) Provisional application No. 63/460,521, filed on Apr. 19, 2023.
(60) Provisional application No. 63/463,415, filed on May 2, 2023.
(60) Provisional application No. 63/623,033, filed on Jan. 19, 2024.

(51) Int. Cl. G16B 20/20; G06N 20/20; G16B 40/20

(52) U.S. Cl. CPC G16B 20/20; G06N 20/20; G16B 40/20

(57) ABSTRACT

Methods, systems, and apparatus, including computer programs encoded on a computer storage medium, for identifying target genes that are predicted to be associated with a disease. In one aspect, a system comprises receiving data identifying: (i) a set of one or more disease phenotypes associated with the disease and (ii) a set of one or more healthy phenotypes that are not associated with the disease, determining a plurality of paths in a phenotype space, that each start from a respective disease phenotype and terminate at a respective healthy phenotype, processing data defining each path in the phenotype space using a phenotype-to-omics machine learning model to generate data defining a corresponding path in an omics space, and processing the data defining the paths in the omics space to identify a set of one or more target genes that are each predicted to be associated with the disease.

### RELATED APPLICATIONS

This application claims priority to U.S. provisional Application No. 63/458,564, filed Apr. 11, 2023, U.S. provisional Application No. 63/460,521, filed Apr. 19, 2023; U.S. provisional application No. 63/463,415, filed May 2, 2023; and U.S. provisional Application No. 63/623,033, filed Jan. 19, 2024; all of which are incorporated herein by reference.

### BACKGROUND

This specification relates to processing data using machine learning models.

Machine learning models receive an input and generate an output, e.g., a predicted output, based on the received input. Some machine learning models are parametric models and generate the output based on the received input and on values of the parameters of the model.

Some machine learning models are deep models that employ multiple layers of models to generate an output for a received input. For example, a deep neural network is a deep machine learning model that includes an output layer and one or more hidden layers that each apply a non-linear transformation to a received input to generate an output.

### SUMMARY

This specification generally describes a system implemented as computer programs on one or more computers in one or more locations that can process embeddings of biological entity-type data, e.g., omics, phenotype, and compound data, using a generative machine learning model (“generative model”) to generate an embedding of different biological entity-type data.

An “embedding” of an entity can refer to an ordered collection of numerical values, e.g., a vector, matrix, or other tensor of numerical values, representing the entity.

The system can create a unified relationship between the omics embedding space (“omics space”), the phenotype embedding space (“phenotype space”), and the compound embedding space (“compound space”), using one or more generative machine learning models. The unified relationship can be used to generatively inform drug discovery.

The system can include an omics embedding model, a phenotype embedding model, and a compound embedding model that can generate respective embeddings by processing omics data, phenotype data, compound data, respectively. The system can also include one or more of: (i) a generative model that maps from the omics space to the phenotype embedding space, (ii) a generative model that maps from the phenotype embedding space to the omics embedding space, or (iii) a bidirectional generative model that can map from the omics embedding space to the phenotype embedding space and from the phenotype embedding space to the omics embedding space. As an example, the bidirectional generative models can be an invertible generative model.

The system can additionally map between the omics space, the phenotype space, and the compound space. In particular, the system can include one or more of: (i) a generative model that maps from the compound embedding space to the omics embedding space, (ii) a generative model that maps from the compound embedding space to the phenotype embedding space, (iii) a generative model that maps from the omics embedding space to the compound embedding space, (iv) a generative model that maps from the phenotype embedding space to the compound embedding space, a (v) a bidirectional generative model that can map from the omics embedding space to the compound embedding space and from the compound embedding space to the omics embedding space, or a (vi) a bidirectional generative model that can map from the phenotype embedding space to the compound embedding space and from the compound embedding space to the phenotype embedding space.

The system can use the one or more generative machine learning models to leverage the associations and interplay between the omics, phenotype, and compound spaces in order to inform drug discovery. As an example, the system can identify one or more target omics states associated with a disease and assess the phenotypic effects of candidate compounds that can affect those target omics states in a subject. As another example, the system can predict a new indication for an existing drug compound, e.g., a new disease that can be treated using the drug compound.

A subject can refer to, e.g., a collection of one or more cells, tissue, or an organism, e.g., an animal or a human.

Omics data can refer to, e.g., genomic data, transcriptomic data, proteomic data, metabolomic data, epigenomic data, or any combination thereof. Genomic data characterizes genetic information of a subject, e.g., the DNA sequence of the subject and gene expression in the subject. Transcriptomic data characterizes RNA transcripts (e.g., mRNA, non-coding RNA) produced by a subject. Proteomic data characterizes the set of proteins produced by a subject. Metabolomic data characterizes metabolites produced by a subject. Epigenomic data characterizes changes in gene expression or function that are not caused by changes in a DNA sequence itself, but by modifications to DNA or the proteins associated with DNA.

In particular, the omics data, e.g., the genomic, transcriptomic, proteomic, metabolomic, epigenomic data, etc., can be represented as a set of omics states in a vector or a corresponding set of vectors. For example, the omics data can be genomic data, e.g., gene expression level data, e.g., where the value of each entry in the vector defines an expression level of the gene in a subject. As another example, the omics data can include transcriptomic data, e.g., protein abundance data, e.g., where the value of each entry in the vector defines a concentration level of a protein biomarker in a subject. As yet another example, the omics data can include metabolomic data, e.g., glucose data, e.g., where the value of each entry in the vector defines a concentration level of glucose in a subject. As yet another example, the omics data can include epigenomic data, e.g., methylation or histone acetylation data, e.g., where the value of each entry in the vector indicates the level of epigenetic modification.

Phenotypes of a subject are the observable physical, behavioral, or biochemical characteristics of the subject. Morphological phenotypes refer to the physical characteristics of an entity, such as shape and size. Physiological phenotypes relate to the function of the organs and systems of a subject, such as heart rate, blood pressure, or hormone levels. Behavior phenotypes characterize the actions or reactions of a subject, such as the response of the subject to stimuli or social behavior of the subject. For instance, human phenotype data can characterize any in vivo phenotype data (i.e., relating in this case to humans rather than animals models), clinical scale data (e.g., characterizing one or more of: apparent sadness, reported sadness, inner tension, reduced sleep, reduced appetite, irritability, aggressiveness, etc., as measured through clinical scales such as Positive and Negative Syndrome Scale (PANSS), Brief Assessment of Cognition in Schizophrenia (BACS)), etc. Imaging phenotypes refer to quantitative or qualitative measures extracted from medical imaging, e.g., images from Magnetic Resonance Imaging (MRI), Computed Tomography (CT), Positron Emission Tomography (PET), microscopy, biopsy, histopathology, etc.

In particular, the phenotype data can be represented as a set of phenotype states represented as one vector or a corresponding set of vectors. For example, the phenotype data can be observable physical traits of a disease, e.g., where the value of each entry in the vector defines a presentation of a specific symptom in a subject. As another example, the phenotype data can be drug response data, e.g., where the value of each entry in the vector defines a change in the presentation of a disease in a subject in response to the administration of a drug. As yet another example, the phenotype data can be imaging data. In this case, the system can further process the imaging data, e.g., using a machine learning model, to determine corresponding values as entries in a vector, e.g., where the value in each entry of the vector characterize tissue function or composition as portrayed in the image.

According to a first aspect there is provided a method for obtaining an embedding of omics data, processing the embedding of the omics data, by a generative machine learning model and in accordance with values of a set of generative machine learning model parameters, to generate an embedding of corresponding phenotype data, and outputting the embedding of the phenotype data.

In some implementations, wherein the generative machine learning model is a bidirectional generative machine learning model that includes an omics-to-phenotype generative machine learning model that is configured to map embeddings of omics data to corresponding embeddings of phenotype data, and a phenotype-to-omics generative machine learning model that is configured to map embeddings of phenotype data to corresponding embeddings of omics data.

In some implementations, the method further includes training the generative machine learning model in accordance with a bidirectionality constraint including processing a first embedding of omics data, by the omics-to-phenotype generative machine learning model and in accordance with values of a set of generative machine learning model parameters, to generate a corresponding embedding of phenotype data, processing the embedding of phenotype data, by the phenotype-to-omics generative machine learning model and in accordance with the values of the set of generative machine learning model parameters, to generate a second embedding of the omics data, and training the generative machine learning model to reduce a discrepancy between the first embedding and the second embedding of the omics data.

In some implementations, wherein the generative machine learning model includes a diffusion model, and further including conditioning the diffusion model on at least one of: a set of omics criteria in omics space or a set of phenotype criteria in phenotype space, and processing perturbations including updates to the omics data in the omics space or updates to the phenotype data in the phenotype space with respect to the set of phenotype criteria or set of omics criteria, respectively.

In some implementations, wherein obtaining the embedding of the omics data includes obtaining the embedding of the omics data using a graph representation of a collection of omics data, wherein the graph representation includes a set of nodes and a set of edges, and wherein: each node in the graph representation is associated with a respective node embedding of each of one or more omics states, and each edge in the graph representation connects a respective pair of nodes in the graph representation and includes a respective edge embedding that represents a relationship between a pair of one or more omics states corresponding to the respective pair of nodes.

In some implementations, wherein the generative machine learning model includes an encoder-decoder model that has been trained to map between a node embedding space and an omics data space.

In some implementations, further including using an omics decoder of the encoder-decoder model to reconstruct omics data from a node embedding in the graph representation.

In some implementations, wherein the graph representation of the collection of omics data is generated using a graph neural network.

In some implementations, wherein generating the graph representation of the collection of omics data using the graph neural network includes generating one or more edge embeddings associated with the edges in the graph representation of omics data and one or more node embeddings associated with nodes in the graph representation of the omics data, including: receiving a set of current edge embeddings associated with the edges and a set of current node embeddings associated with the nodes in the graph representation, and updating the current edge embeddings and current node embeddings by performing message passing operations that are conditioned on a topology of the graph representation and are parametrized by a set of graph network layer parameters.

In some implementations, wherein updating the current edge embeddings and current node embeddings further includes comparing a node embedding of a first omics state and a node embedding of a second omics state from the graph representation using a measure of similarity to generate a similarity score between the first omics state and the second omics state, and using the similarity score to predict an edge between the first omics state and the second omics state in the graph representation.

In some implementations, further including identifying, based on the embedding of the phenotype data, one or more candidate omics states, obtaining experimental data characterizing each of the one or more candidate omics states, and updating the values of the set of generative machine learning model parameters based on the experimental data using a machine learning training technique.

In some implementations, wherein the omics data includes one or more of: genomic data, or proteomic data, or transcriptomic data, or epigenomic data, or metabolomic data.

In some implementations, wherein the phenotype data includes one or more of: morphological phenotype data, or physiological phenotype data, or biochemical phenotype data, or behavioral phenotype data.

In some implementations, wherein the generative machine learning model is trained through active learning techniques, including evaluating a measure of importance indicative of relevance to a disease of a region in at least one of omics or phenotype space, obtaining experimental data in accordance with an identified region of high importance, and incorporating the experimental data into the graph representation using the graph neural network to add a new edge or node to the graph representation.

In some implementations, further including receiving data characterizing a compound, processing the data characterizing the compound using a compound embedding model to generate an embedding of the compound, and conditioning the generative machine learning model on the embedding of the compound.

In some implementations, wherein conditioning the generative machine learning model on the embedding of the compound includes processing the embedding of the omics data in accordance with the values of the set of generative machine learning model parameters and the embedding of the compound to generate an embedding of corresponding phenotype data indicative of a particular phenotype predicted to arise from the compound being administered to a subject.

In some implementations, further including generating data defining a path through phenotype space, and processing the data defining the path through phenotype space, using a generative machine learning model, to generate a corresponding path through omics space.

In some implementations, wherein generating data defining the path through phenotype space includes generating the data defining the path through phenotype space to optimize a cost function that measures a cost of a corresponding path through omics space.

In some implementations, wherein the path through phenotype space starts from a disease phenotype state and terminates at a healthy phenotype state.

In some implementations, wherein the path through phenotype space includes a phenotype state represented by the embedding of phenotype data generated by processing the embedding of the omics data.

In some implementations, further including identifying one or more biomarkers corresponding with the one or more omics states using the phenotype state.

In some implementations, further including identifying one or more drug compounds that target one or more omics states along the path through omics space.

In some implementations, further including determining that one or more of the drug compounds should be administered to a subject.

In some implementations, further including administering one or more of the drug compounds to the subject.

According to a second aspect there is provided a method for identifying one or more target genes that are predicted to be associated with a disease, the method including: receiving data identifying: (i) a set of one or more disease phenotypes associated with the disease, and (ii) a set of one or more healthy phenotypes that are not associated with the disease, determining a plurality of paths, in a phenotype space, that each start from a respective disease phenotype in the set of one or more disease phenotypes and terminates at a respective healthy phenotype in the set of healthy phenotypes, processing data defining each path in the phenotype space, using a phenotype-to-omics machine learning model and in accordance with values of a set of model parameters of the phenotype-to-omics machine learning model, to generate data defining a corresponding path in an omics space, and processing the data defining the paths in the omics space to identify a set of one or more target genes that are each predicted to be associated with the disease.

In some implementations, further including processing the data defining the paths in the omics space to identify one or more candidate biomarkers that are each predicted to be associated with the disease.

In some implementations, wherein processing the data defining the paths in the omics space to identify a set of one or more target genes that are each predicted to be associated with the disease includes determining, for each path in the omics space and for each gene in a set of genes, a respective variation in an expression level of the gene along the path in the omics space, and identifying the set of target genes based on the variations in expression level of the genes in the set of genes along the paths in omics space.

In some implementations, wherein identifying the set of target genes based on the variations in expression level of the genes in the set of genes along the paths in omics space includes identifying one or more genes associated with highest variations in expression level as being target genes.

In some implementations, wherein the set of one or more target genes includes a plurality of target genes.

In some implementations, further including determining a ranking of the plurality of target genes, including determining, for each of the plurality of target genes, a plurality of gene-specific paths in the omics space that each start from an omics state having a first expression level for the target gene and terminate at an omics state having a second, lower expression level for the target gene, determining the ranking of the plurality of target genes based on the plurality of gene-specific paths.

In some implementations, wherein determining the ranking of the plurality of target genes based on the gene-specific paths includes determining a respective score for each of the plurality of target genes, including, for each target gene processing data defining each gene-specific path for the target gene in the omics space, using an omics-to-phenotype machine learning model in accordance with values of a set of omics-to-phenotype machine learning model parameters, to generate data defining a corresponding gene-specific path in the phenotype space, determining, for each gene-specific path in the phenotype space, a path classification that classifies whether the gene-specific path terminates at a healthy phenotype, and determining the score the target gene based on the path classifications for the gene-specific paths for the target gene in the phenotype space, and determining the ranking of the plurality of target genes based on the scores for the target genes.

In some implementations, wherein determining the score for the target gene based on the path classifications for the gene-specific paths for the target gene in the phenotype space includes determining the score for the target gene based on a fraction of gene-specific paths for the target gene that are classified as terminating at a healthy phenotype.

In some implementations, wherein the phenotype-to-omics machine learning model is implemented as a generative diffusion model.

In some implementations, wherein the phenotype-to-omics machine learning model has been trained on a set of training examples by a machine learning training technique, wherein each training example corresponds to a respective subject and characterizes: (i) a phenotype state of the subject, and (ii) an omics state of the subject.

In some implementations, further including identifying a drug that targets a target gene in the set of one or more target genes, and determining that a patient, having the disease, should receive the identified drug.

In some implementations, further including administering the identified drug to the patient.

In some implementations, further including selecting a target gene from the set of one or more target genes that are predicted to be associated with the disease, identifying one or more drug compounds that are associated with the target gene, determining, based on the one or more drug compounds that are associated with the target gene, a set of one or more criteria specifying one or more properties that are predicted to be satisfied by drug compounds that act on the target gene, identifying a plurality of compounds that satisfy the set of one or more criteria that are predicted to be satisfied by drug compounds that act on the target gene, obtaining, for each of the plurality of compounds, experimental data that characterizes an omics perturbation resulting from administering the drug compound to a subject, training a compound-to-omics machine learning model using the experimental data, wherein the compound-to-omics machine learning model is configured to process a model input that characterizes a compound to generate a predicted omics perturbation that would result from administering the drug compound to a subject, and identifying one or more candidate drug compounds for the disease using the compound-to-omics machine learning model.

In some implementations, wherein identifying a plurality of compounds that satisfy the set of one or more criteria that are predicted to be satisfied by drug compounds that act on the target gene includes sampling the plurality of compounds from a space of compounds that satisfy the set of one or more criteria.

In some implementations, wherein sampling the plurality of compounds from a space of compounds that satisfy the set of one or more criteria includes generating one or more of the plurality of compounds by applying a modification to a respective one of the drug compounds that are associated with the target gene.

In some implementations, wherein sampling the plurality of compounds from a space of compounds that satisfy the set of one or more criteria includes determining, for each of the plurality of compounds, a set of one or more properties of the compound including a predicted binding affinity of the compound for a protein coded for by the target gene, and filtering the plurality of compounds in accordance with filtering criteria based on the properties of the compounds.

In some implementations, wherein identifying one or more candidate drug compounds for the disease using the compound-to-omics machine learning model includes generating one or more candidate drug compounds using the properties of the compounds, determining, for each of the one or more candidate drug compounds and using the compound-to-omics machine learning model, a predicted omics perturbation that would result from administering the candidate drug compound to a subject.

In some implementations, further including, after identifying the one or more candidate drug compounds for the disease using the compound-to-omics machine learning model, obtaining, for each of the candidate drug compounds, experimental data that characterizes an omics perturbation resulting from administering the candidate drug compound to a subject, and training the compound-to-omics machine learning model using the experimental data for the candidate drug compounds.

In some implementations, further including obtaining, for each of the plurality of compounds, experimental data that characterizes a phenotype perturbation resulting from administering the drug compound to a subject, and training an omics-to-phenotype machine learning model using the experimental data, wherein the omics-to-phenotype machine learning model is configured to process a model input that characterizes an omics state to generate a predicted phenotype state corresponding to the omics state.

In some implementations, wherein identifying one or more candidate drug compounds for the disease using the compound-to-omics machine learning model includes determining, for each of the one or more candidate drug compounds and using the compound-to-omics machine learning model, a predicted omics perturbation that would result from administering the candidate drug compound to a subject, and determining, for each of the one or more candidate drug compounds and using the omics-to-phenotype machine learning model, a predicted phenotype perturbation that would result from administering the candidate drug compound to the subject.

In some implementations, wherein the predicted phenotype perturbation includes a predicted outcome of an assay performed to determine a response from administering the candidate drug compound to the subject.

In some implementations, wherein the compound-to-omics machine learning model is implemented as a generative diffusion model.

According to a third aspect there is provided a method for receiving data identifying a target gene that is predicted to be associated with a disease, identifying one or more drug compounds that are associated with the target gene, determining, based on the one or more drug compounds that are associated with the target gene, a set of one or more criteria specifying one or more properties that are predicted to be satisfied by drug compounds that act on the target gene, identifying a plurality of compounds that satisfy the set of one or more criteria that are predicted to be satisfied by drug compounds that act on the target gene, obtaining, for each of the plurality of compounds, experimental data that characterizes an omics perturbation resulting from administering the drug compound to a subject, training a compound-to-omics machine learning model using the experimental data, wherein the compound-to-omics machine learning model is configured to process a model input that characterizes a compound to generate a predicted omics perturbation that would result from administering the drug compound to a subject, and identifying one or more candidate drug compounds for the disease using the compound-to-omics machine learning model.

In some implementations, wherein identifying a plurality of compounds that satisfy the set of one or more criteria that are predicted to be satisfied by drug compounds that act on the target gene includes sampling the plurality of compounds from a space of compounds that satisfy the set of one or more criteria.

In some implementations, wherein sampling the plurality of compounds from a space of compounds that satisfy the set of one or more criteria includes generating one or more of the plurality of compounds by applying a modification to a respective one of the drug compounds that are associated with the target gene.

In some implementations, wherein sampling the plurality of compounds from a space of compounds that satisfy the set of one or more criteria includes determining, for each of the plurality of compounds, a set of one or more properties of the compound including a predicted binding affinity of the compound for a protein coded for by the target gene, and filtering the plurality of compounds in accordance with filtering criteria based on the properties of the compounds.

In some implementations, wherein identifying one or more candidate drug compounds for the disease using the compound-to-omics machine learning model includes generating one or more candidate drug compounds using the properties of the compounds, determining, for each of the one or more candidate drug compounds and using the compound-to-omics machine learning model, a predicted omics perturbation that would result from administering the candidate drug compound to a subject.

In some implementations, further including, after identifying the one or more candidate drug compounds for the disease using the compound-to-omics machine learning model, obtaining, for each of the candidate drug compounds, experimental data that characterizes an omics perturbation resulting from administering the candidate drug compound to a subject, and training the compound-to-omics machine learning model using the experimental data for the candidate drug compounds.

In some implementations, further including obtaining, for each of the plurality of compounds, experimental data that characterizes a phenotype perturbation resulting from administering the drug compound to a subject, and training an omics-to-phenotype machine learning model using the experimental data, wherein the omics-to-phenotype machine learning model is configured to process a model input that characterizes an omics state to generate a predicted phenotype state corresponding to the omics state.

In some implementations, wherein identifying one or more candidate drug compounds for the disease using the compound-to-omics machine learning model includes determining, for each of the one or more candidate drug compounds and using the compound-to-omics machine learning model, a predicted omics perturbation that would result from administering the candidate drug compound to a subject, determining, for each of the one or more candidate drug compounds and using the omics-to-phenotype machine learning model, a predicted phenotype perturbation that would result from administering the candidate drug compound to the subject.

In some implementations, wherein the predicted phenotype perturbation includes a predicted outcome of an assay performed to determine a response from administering the candidate drug compound to the subject.

In some implementations, wherein the compound-to-omics machine learning model is implemented as a generative diffusion model.

In some implementations, further including physically synthesizing a candidate drug compound identified using the compound-to-omics machine learning model.

In some implementations, further including performing physical experiments to test one or more properties of the synthesized candidate drug.

In some implementations, further including determining that a subject should receive a candidate drug compound identified using the compound-to-omics machine learning model in order to treat the disease.

In some implementations, wherein receiving data identifying the target gene that is predicted to be associated with the disease includes receiving data identifying: (i) a set of one or more disease phenotypes associated with the disease, and (ii) a set of one or more healthy phenotypes that are not associated with the disease, determining a plurality of paths, in a phenotype space, that each start from a respective disease phenotype in the set of one or more disease phenotypes and terminates at a respective healthy phenotype in the set of healthy phenotypes, processing data defining each path in the phenotype space, using a phenotype-to-omics machine learning model and in accordance with values of a set of model parameters of the phenotype-to-omics machine learning model, to generate data defining a corresponding path in an omics space, and processing the data defining the paths in the omics space to identify a set of one or more target genes that are each predicted to be associated with the disease.

In some implementations, wherein processing the data defining the paths in the omics space to identify a set of one or more target genes that are each predicted to be associated with the disease includes determining, for each path in the omics space and for each gene in a set of genes, a respective variation in an expression level of the gene along the path in the omics space, and identifying the set of target genes based on the variations in expression level of the genes in the set of genes along the paths in omics space.

In some implementations, identifying the set of target genes based on the variations in expression level of the genes in the set of genes along the paths in omics space includes identifying one or more genes associated with highest variations in expression level as being target genes.

In some implementations, wherein the set of one or more target genes includes a plurality of target genes.

In some implementations, further including determining a ranking of the plurality of target genes, including determining, for each of the plurality of target genes, a plurality of gene-specific paths in the omics space that each start from an omics state having a first expression level for the target gene and terminate at an omics state having a second, lower expression level for the target gene, and determining the ranking of the plurality of target genes based on the plurality of gene-specific paths.

In some implementations, wherein determining the ranking of the plurality of target genes based on the gene-specific paths includes determining a respective score for each of the plurality of target genes, including, for each target gene, processing data defining each gene-specific path for the target gene in the omics space, using an omics-to-phenotype machine learning model in accordance with values of a set of omics-to-phenotype machine learning model parameters, to generate data defining a corresponding gene-specific path in the phenotype space, determining, for each gene-specific path in the phenotype space, a path classification that classifies whether the gene-specific path terminates at a healthy phenotype, and determining the score the target gene based on the path classifications for the gene-specific paths for the target gene in the phenotype space, and determining the ranking of the plurality of target genes based on the scores for the target genes.

In some implementations, wherein determining the score for the target gene based on the path classifications for the gene-specific paths for the target gene in the phenotype space includes determining the score for the target gene based on a fraction of gene-specific paths for the target gene that are classified as terminating at a healthy phenotype.

In some implementations, wherein the phenotype-to-omics machine learning model is implemented as a generative diffusion model.

In some implementations, wherein the phenotype-to-omics machine learning model has been trained on a set of training examples by a machine learning training technique, wherein each training example corresponds to a respective subject and characterizes: (i) a phenotype state of the subject, and (ii) an omics state of the subject.

According to a fourth aspect there is provided a method for receiving data identifying a drug compound associated with a first indication, processing a model input that characterizes the drug compound using a compound-to-omics machine learning model, wherein the compound-to-omics machine learning model is configured to process the model input to generate a predicted omics perturbation that would result from administering the drug compound to a subject, to generate a corresponding set of omics perturbations for the drug compound, applying the set of omics perturbations to a set of initial omics states to generate a set of perturbed omics states, processing the set of perturbed omics states using an omics-to-phenotype machine learning model, wherein the omics-to-phenotype machine learning model is configured to process a model input that characterizes an omics state to generate a predicted phenotype state corresponding to the omics state, to generate a corresponding set of predicted perturbed phenotype states for the drug compound, determining a first distribution of phenotype states corresponding with the set of initial omics states and a second distribution of phenotype states using the predicted perturbed phenotype states, and identifying a predicted indication for the compound using a determined discrepancy between the first and second distributions of phenotype states in accordance with a phenotypic indicator.

In some implementations, wherein receiving data identifying a compound associated with a first indication includes receiving an embedding of a compound associated with a target gene that is predicted to be associated with a disease.

In some implementations, wherein processing the model input that characterizes the drug compound using a compound-to-omics machine learning model includes processing an embedding of compound data.

In some implementations, further including determining a likelihood of the predicted omics perturbation, wherein determining the likelihood of the predicted omics perturbation includes processing an intermediate output of the compound-to-omics machine learning model to output a corresponding likelihood of the predicted omics perturbation.

In some implementations, wherein the corresponding set of omics perturbations for the drug compound includes a top N omics perturbations for the drug compound based at least on the predicted likelihood of the predicted omics perturbation.

In some implementations, wherein the corresponding set of omics perturbations for the drug compound includes a random sample of omics perturbations for the drug compound from a subset of possible omics perturbations, wherein the subset includes the omics perturbations with a corresponding likelihood greater than a threshold.

In some implementations, wherein the compound-to-omics machine learning model includes an ensemble of compound-to-omics machine learning models that have been trained with one or more of different initializations or different architectures.

In some implementations, further including generating a plurality of omics perturbations using the ensemble of compound-to-omics machine learning models as the set of omics perturbations for the drug compound.

In some implementations, further including using dropout regularization to generate a plurality of omics perturbations as the set of omics perturbations by processing the drug compound a corresponding plurality of prediction iterations, wherein each prediction iteration is characterized by a first dropout rate.

In some implementations, further including receiving the set of initial omics states.

In some implementations, receiving the set of initial omics states further includes identifying a set of phenotypes of interest, and processing the set of phenotypes of interest using a phenotype-to-omics machine learning model, wherein the phenotype-to-omics machine learning model is configured to process a model input that characterizes phenotype data to generate corresponding omics data, to generate the set of initial omics states.

In some implementations, wherein applying the set of omics perturbations to the set of initial omics states to generate a set of perturbed omics states further includes, for each predicted omics perturbation and each initial omics state, applying an embedding that characterizes the predicted omics perturbation to an embedding that characterizes the initial omics state, and generating a corresponding perturbed omics state embeddings.

In some implementations, wherein processing the set of perturbed omics states for the drug compound further includes processing a set of the perturbed omics state embeddings.

In some implementations, wherein determining a first distribution of phenotype states corresponding with the set of initial omics states includes processing the set of initial omics state using the omics-to-phenotype machine learning model to generate the corresponding set of phenotype states.

In some implementations, wherein identifying a predicted indication for the compound using the determined discrepancy between the first and second distributions of phenotype states in accordance with the phenotypic indicator includes determining a measure of discrepancy between each initial phenotype and predicted perturbed phenotype state pair in accordance with the phenotypic indicator, wherein each phenotype state pair includes an initial phenotype state associated with a respective initial omics state and a predicted perturbed phenotype state associated with a respective perturbed omics state that represents applying a respective omics perturbation to the initial omics state, and determining that at least a first threshold number of phenotype state pairs have a measure of discrepancy greater than a second threshold for the phenotypic indicator.

In some implementations, wherein determining the measure of the discrepancy between each initial phenotype and predicted perturbed phenotype state pair in accordance with the phenotypic indicator includes determining a measure of distance between an embedding of the initial phenotype and an embedding of the perturbed phenotype state with respect to a dimension corresponding with the phenotypic indicator.

In some implementations, wherein determining a measure of discrepancy between each initial phenotype and predicted perturbed phenotype state pair in accordance with the phenotypic indicator includes, for each phenotype state pair, determining a first measure of similarity between an embedding of the initial phenotype state in the phenotype state pair and an embedding of a target phenotype indicative of the phenotypic indicator in accordance with an inner product of embeddings, and determining a second measure of similarity between an embedding of the predicted perturbed phenotype state in the phenotype state pair and the embedding of the target phenotype indicative of the phenotypic indicator in accordance with an inner product of embeddings.

In some implementations, further including determining that that a discrepancy between the second measure and the first measure exceeds a first threshold number for a second number of phenotype state pairs.

In some implementations, wherein each predicted perturbed phenotype state includes a predicted outcome of an assay performed to determine a response from administering the drug compound to a subject.

In some implementations, wherein the compound-to-omics machine learning model is implemented as a generative diffusion model.

In some implementations, further including obtaining the drug compound, and performing physical experiments to test one or more properties of the drug compound with respect to the identified predicted indication.

In some implementations, further including determining that the drug compound should be administered to a subject for treating the predicted indication.

In some implementations, further including administering the drug compound to a subject for treating the predicted indication.

Particular embodiments of the subject matter described in this specification can be implemented so as to realize one or more of the following advantages.

The techniques described in this specification enable target discovery through in-silico prediction for a disease. In particular, the system provides the ability to jointly optimize generative models that can map between omics, phenotype, and compound spaces in order to efficiently learn and exploit relationships between the spaces. These relationships can be used to identify target omics states based on a change in gene expression between a disease phenotype and a healthy phenotype as well as drug compounds that target identified target omics states.

More specifically, certain omics states (or transitions between omics states or regions of omics space) can be associated with particular compounds (e.g., drugs, e.g., from a library of drug molecules). For instance, a compound may be associated with a change in proteomics expression in a subject, or with a particular genomics state of a subject. The system can determine a path of phenotypes, e.g., by perturbing a phenotype state, can generate a corresponding path through the omics space (e.g., using the phenotype-to-omics model), and then identify one or more compounds that are associated with the path through the omics space. The system can select the path through the phenotype space as one that transitions from a diseased phenotype to a healthy phenotype, and the system can then identify one or more compounds that are associated with that transition. The identified compounds can provide a starting point for drug discovery, or for repurposing existing compounds in order to treat different diseases.

Rather than relying on computationally expensive grid searches through all possible combinations of omics-phenotype, omics-compound, and phenotype-compound relationships, the system can leverage the information, e.g., experimental data and actively learned data, in the respective embedding spaces in order to inform predicted results. As an example, the system can model a region of relevant compounds to inform drug discovery with respect to characteristics of those compounds, thereby decreasing the computational resources necessary to perform the prediction by restricting the space of compounds.

More specifically, instead of directly storing intractable numbers of omics-phenotype, omics-compound, and phenotype-compound tuples, the system can model the relationships using the parameters of each respective generative machine learning model to implicitly store data representing each of the tuples in a much more efficient manner, thereby reducing computational resources. In particular, the system can train a generative model with one or more orders of magnitude fewer parameters to implicitly store the tuples by representing a transformation relationship between each pair of respective spaces.

The system can also explore the relationships between each pair of respective spaces to accelerate drug repurposing, e.g., by predicting indications for existing drugs. In particular, the system can leverage the relationships between the compound and omics space and the omics and phenotype space to predict new indications for a drug compound, e.g., using the omics space as an intermediary to connect between phenotype and compound space. In particular, the system can be used to probe the predicted impact of a particular drug compound on a number of initial omics states, e.g., outside of effects corresponding with a known disposition. The system can then evaluate a criterion for identifying a new indication, e.g., using a discrepancy between the initial phenotype states and the predicted perturbed phenotype states in phenotype space to determine whether the drug compound is predicted to be associated with a different indication.

Furthermore, the system can circumvent the need to use computationally intensive statistical analysis and clustering operations to process massive high-dimensional feature dataset for large populations of subjects in order to predict indications for existing drugs or identify target genes associated with a particular disease. In contrast, the system can leverage the generative machine learning models in a manner that consumes significantly fewer resources, e.g., because the generative machine learning models can be trained to simply process data corresponding to a state or a path, e.g., a path in phenotype space, to generate a corresponding state or path in a different biological entity space, e.g., a path in omics space, which is much smaller relative to a massive high-dimensional feature dataset for a large population of subjects.

Additionally, the system can allow for efficient compound modelling that does not rely on explicitly calculating a large number of possible atom positions and bonds between those atoms, e.g., which can be modelled through an optimization in a combinatorically large space. While current compound modeling approaches can require extremely large combinatoric calculations, the system as described provides techniques for jointly optimizing a compound-to-omics and omics-to-phenotype model to enable the identification of potential candidates without the burden of searching the entire space of possible compounds. For example, by identifying a specific region of interest in compound space and restricting the region of space where the compound-to-omics model is trained to operate, e.g., to the region of interest, the system can require less training data and fewer training iterations in order to accurately model compounds in an appropriate region for drug discovery. More specifically, the system can analyze docking patterns in compound space to identify candidate compounds, predict perturbations in omics space from administering the candidate compounds using the compound-to-omics model, and predict corresponding perturbations in phenotype space using the omics-to-phenotype model.

Moreover, the techniques of this specification can reduce consumption of physical laboratory resources, e.g., equipment, compounds, reagents, personnel hours, and so forth. In particular, rather than running a prohibitive amount of successive assays to evaluate phenotype states resulting from an omics state perturbation, the system can be used to predict results from an assay that can be leveraged for drug discovery. In particular, at the cellular level, a disease can be modeled as a combination of phenotypes of interest, e.g., lysosomal function, mitochondrial function, etc. Experimental validation on each individual gene (or combination of genes) on the entire druggable genome may be infeasible, e.g., as a result of being excessively time consuming and labor intensive.

Furthermore, the system can provide the flexibility to discover biomarkers as well as target omics states, e.g., to identify candidate biomarkers. In particular, the system can analyze how biomarkers change along the different paths in omics space. In some cases, identifying a biomarker along a pathway can lead to the identification of a target omics state, e.g., through informed experimentation.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an overview of an example omics, phenotype, and compound unification system that includes one or more generative models and one or more embedding models.

FIG. 2 illustrates an example biological data graph, including node and edge embeddings that can be used to train the one or more embedding models of FIG. 1.

FIG. 3 is a flow diagram of an example process for outputting an embedding of a different type of biological data using a generative model.

FIG. 4 is a flow diagram of an example process for training a generative model using active learning.

FIG. 5 illustrates how the system can identify an example path from a disease state to a healthy state that can be informed by optimizing a cost function.

FIG. 6 is a flow diagram of an example process for conditioning a generative machine learning model on omics, phenotype, or compound data.

FIG. 7 illustrates an example of identifying decorator molecules that can inform drug discovery using identified paths from disease states to healthy states.

FIG. 8 is a flow diagram of an example process for identifying target genes associated with a disease using paths in phenotype and omics space.

FIG. 9 is a flow diagram of an example process for ranking target genes using the corresponding paths in phenotype and omics space.

FIG. 10 is a flow diagram of an example process for identifying candidate drug compounds for an identified target gene.

FIG. 11 is a block diagram that illustrates an example of using the system to predict new indications of a drug compound.

FIG. 12 illustrates an example of determining a discrepancy between a distribution of predicted perturbed phenotype states and initial phenotype states to identify a predicted indication of a drug compound.

FIG. 13 is a flow diagram of an example process for predicting a new indication of a drug compound.

Like reference numbers and designations in the various drawings indicate like elements.

### DETAILED DESCRIPTION

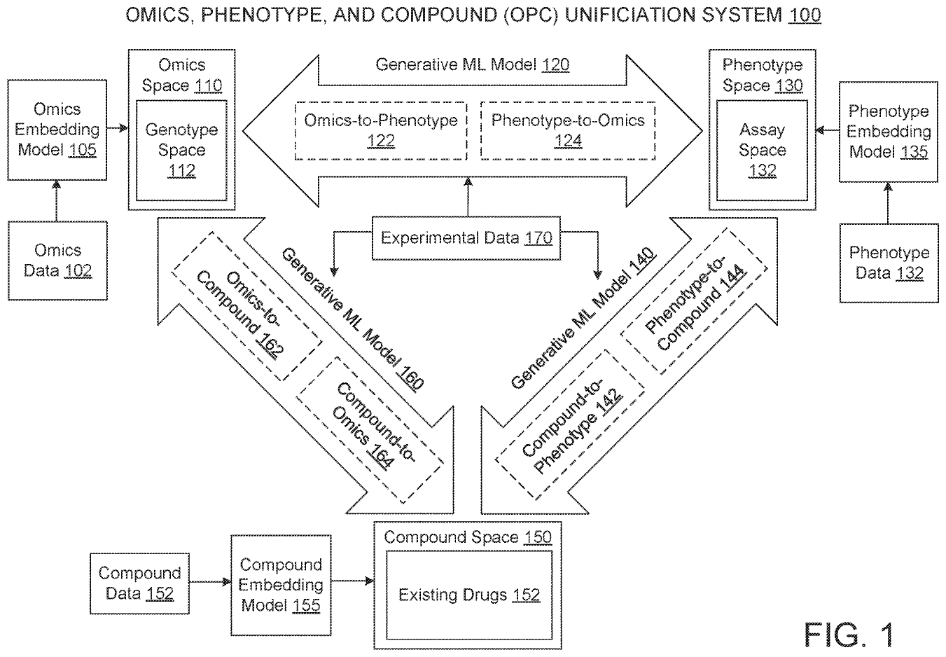

FIG. 1 shows an overview of an example omics, phenotype, and compound (OPC) unification system 100. The OPC unification system 100 is an example of a system implemented as computer programs on one or more computers in one or more locations in which the systems, components, and techniques described below are implemented.

The OPC unification system 100 can map between omics space 110, phenotype space 130, and compound space 150 using one or more generative machine learning models (generative models), e.g., the models 120, 140, and 160. In particular, each generative machine learning model can process an input in accordance with values of a set of machine learning model parameters to generate samples from a distribution over a space of possible outputs, e.g., a different space of outputs than the input space. Each generative machine learning model can be, e.g., a diffusion model, or a variational autoencoder, or a generative adversarial neural network, or a normalizing flow, or any other appropriate generative machine learning model that can map between a pairing of the omics, phenotype, and compound spaces.

For example, the model 120 can process, in accordance with values of a set of model 120 parameters, omics data to generate samples from a corresponding distribution over phenotype space, can process phenotype data in accordance with values of a set of model 120 parameters to generate samples from a corresponding distribution over omics space, or both. As another example, the model 140 can process, in accordance with values of a set of model 140 parameters, phenotype data to generate samples from a corresponding distribution over compound space, compound data to generate samples from a corresponding distribution over phenotype space, or both. As yet another example, the model 160 can process, in accordance with values of a set of model 160 parameters, omics data to generate samples from a corresponding distribution in compound space, compound data to generate samples from a corresponding distribution in omics space, or both.

As an example, each of the generative machine learning models 120, 140, and 160 can include a bidirectional model that maps between two spaces, e.g., the omics and phenotype space, the omics and compound space, or the phenotype and compound space. As another example, each of the generative machine learning models 120, 140, and 160 can include a pair of corresponding individual models that map between two spaces in opposite directions, e.g., the omics-to-phenotype model 122 and the phenotype-to-omics model 124, the compound-to-phenotype model 142 and the phenotype-to-compound 144, or the-omics-to-compound model 162 and the compound-to-omics model 164.

More specifically, mapping between two spaces, e.g., a first space and a second different space, in this context refers to processing an embedding of data in the first space, e.g., an omics space 110, phenotype space 130, or compound space 150, to generate a corresponding embedding of data in the second different space, e.g., the omics space 110, phenotype space 130, or the compound space 150. In particular, mapping can refer to processing an embedding including a number of different omics states, e.g., genes, phenotype states, e.g., phenotypes, or compound states, e.g., compounds, to generate a corresponding embedding of data in a different space, as will be discussed in more detail below.

The models 120, 140, and 160 rely on the creation of initial embeddings in the omics space 110, phenotype space 130, and compound space 150, respectively. In particular, the OPC unification system 100 can additionally include embedding models, e.g., the omics embedding model 105, the phenotype embedding model 135, and the compound embedding model 155, to embed omics data, phenotype data, and compound data into omics 110, phenotype 130, and compound spaces 150, respectively. For instance, the omics space 110, phenotype space 130, and compound space 150 can have 10 dimensions, or 100 dimensions, or 1000 dimensions, or any other appropriate number of dimensions.

More specifically, the omics embedding model 105 can be configured to process omics data 102 to generate an embedding of the omics data in a latent space, e.g., the omics space 110. In some cases, the omics space 110 is a genotype space 112. The omics data can be, e.g., genomic data, proteomic data, metabolomic data, transcriptomic data, epigenomic data, or any other appropriate “omics” data. In some cases, the omics space 110 can be a multi-modal space, e.g., where the omics embedding model can be configured to process data characterizing a set of multiple omics modalities to generate a joint embedding in a combined embedding space.

The phenotype embedding model 135 can be configured to process data characterizing a phenotype to generate an embedding of the phenotype in a phenotype latent space 130. In some cases, the phenotype space 110 is an assay space 132, e.g., a space containing data from experiments used to test or determine the presence or concentration of one or more particular molecules in a subject sample. A phenotype can be characterized by, e.g., in vitro phenotype data, in vivo phenotype data, human phenotype data, or a combination thereof. In particular, the phenotype space 130 can be a multi-modal phenotype space that jointly characterizes multiple types of phenotype data, e.g., morphological, physiological, biochemical, or behavior phenotypes measured in vitro, in vivo, or in a human model. The phenotype embedding model 135 can be configured to process single-modality or multi-modal phenotype data to generate a corresponding embedding in the latent space 130.

As an example, in vitro phenotype data can characterize any observable characteristics of cells or tissues that are grown or studied outside of their natural environment, e.g., in a laboratory setting. For instance, in vitro phenotype data can characterize one or more of: cell morphology (e.g., the size, shape, and structure of cells), growth rate (e.g., the rate at which cells divide and proliferate), drug response (e.g., the response of cells or tissues to drugs), cell signaling (e.g., specific signaling pathways observed in cells or tissues), etc.

As another example, in vivo phenotype data can characterize any observable characteristics of living animal models, e.g., rodent models, cat models, pig models, etc. For instance in vivo phenotype data can characterize one or more of: body weight, behavioral responses, vital signs (e.g., heart rate, blood pressure, respiration rate, etc.), imaging data (e.g., x-ray data, computed tomography (CT) data, magnetic resonance imaging (MRI) data, functional MRI (fMRI) data, ultrasound (US) data, etc.), blood markers (e.g., levels of different molecules or cells in the blood), organ function (e.g., as measured through biopsies or functional imaging), etc.

As a further example, human phenotype data can characterize any observable characteristics of individual humans. For instance, human phenotype data can characterize any in vivo phenotype data (i.e., relating in this case to humans rather than animals models), clinical scale data (e.g., characterizing one or more of: apparent sadness, reported sadness, inner tension, reduced sleep, reduced appetite, irritability, aggressiveness, etc., as measured through clinical scales such as Positive and Negative Syndrome Scale (PANSS), Brief Assessment of Cognition in Schizophrenia (BACS)), etc.

The compound embedding model 155 can be configured to process compound data 152 to generate an embedding in compound space 150. The compound space 150 can be a space of possible compounds (including compounds corresponding to existing drugs). A compound can be digitally represented, e.g., by being characterized by elemental analysis, density, solubility, melting and boiling points, molecular structure, e.g., data defining a respective position of each atom in a three-dimensional (3D) atomic structure of the drug, or any combination thereof. As an example, the compound data can be represented, e.g., as a Simplified Molecular Input Line Entry System (SMILES) string, or a graph, e.g., a graph in which each node in the graph represents a respective atom in the molecule and each edge represents a respective bond in the molecule.

Each embedding model 105, 135, and 155 can have any appropriate model architecture that can embed the omics data 102, phenotype data 132, and compound data 152 into respective omics space 110, phenotype space 130, and compound space 150. As an example, in the case that the spaces 110, 130, and 150 are a continuous space, e.g., a Euclidean space, the embedding models 105, 135, 155 can be feedforward neural networks, e.g., including a sequence of fully connected layers. As another example, the embeddings can be generated by a predefined, non-learned procedure, e.g., the system can use one-hot embedding techniques to generate categorical embeddings.

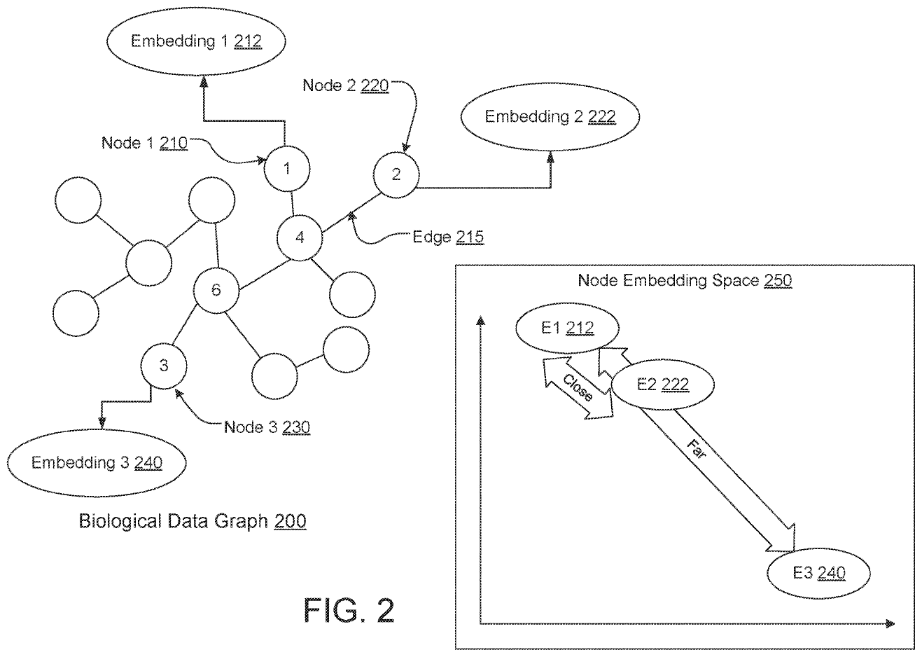

As yet another example, the spaces 110, 130, and 150 can be represented as a graph representation e.g., the spaces can be represented as a graph of discrete states. For example, each of the spaces 110, 130, 150 can be a knowledge graph, where each node in the graph represents a respective omics, phenotype, or compound state, In particular, the system 100 can leverage a biological data graph (e.g., a knowledge graph) that includes nodes corresponding to respective states. Each node in the graph can be associated with an embedding of the particular biological entity state. In this case, one or more of the embedding models 105, 135, 155 can be implemented as a graph neural network or encoder neural network, as will be discussed in more detail in FIG. 2.

In the particular example depicted, the generative machine learning (ML) model 120 is a bidirectional model that maps between an omics space 110 and a phenotype space 130, the generative ML model 140 is a bidirectional model that maps between the phenotype space 130 and a compound space 150, and the generative ML model 160 is a bidirectional model that maps between the omics space 110 and the compound space 150.

As an example, the model 120 can be a generative machine learning model trained to process an embedding of phenotype data in the phenotype space 130 to generate an embedding of omics data in the omics space 110 characterizing a corresponding set of omics states. As another example, the model 140 can be a generative machine learning model trained to process an embedding of omics data in the omics space 110 to generate an embedding of compound data in compound space 150 characterizing a corresponding set of compounds that target the omics states.

In particular, one or more of the generative machine learning models 120, 140, and 160 can be invertible generative models. As an example, the generative model can include, e.g., a diffusion model or an invertible neural network. More specifically, invertibility of the generative model can enable the system to move seamlessly between the forward and the inverse problem, e.g., the first and second space.

In some cases, the generative models 120, 140, and 160 can include corresponding pairs of models to achieve the mapping between the omics space 110 and the phenotype space 130, the phenotype space 130 and the compound space 150, and the omics space 110 and the compound space, respectively. For example, the generative model 120 can include an omics-to-phenotype model 122 and a phenotype-to-omics model 124. As another example, the generative model 140 can include a compound-to-phenotype model 142 and a phenotype-to-compound model 144. As yet another example, the generative model 160 can include an omics-to-compound model 162 and a compound-to-omics model 164. In this case, each model 122, 124, 142, 144, 162, 164 can be a generative machine learning model, e.g., a diffusion model, a variational autoencoder, a generative adversarial neural network, a normalizing flow, or any other appropriate generative machine learning model trained to map between the appropriate first space and the appropriate second space.

In particular, each model pair, e.g., the models 122, 124, models 142, 144, and models 162, 164 can be jointly trained to satisfy a constraint, e.g., a recovery constraint, that mapping a point in the first space to the second space, and mapping the resultant point back to the first space recovers the original point. For example, the phenotype-to-omics 122 and omics-to-phenotype 124 models can be jointly trained to ensure that mapping a point, e.g., an original phenotype state, from the phenotype space 130 to the omics space 110 using the phenotype-to-omics model 124, and mapping the resultant point back from omics space 110 to phenotype space 130 using the omics-to-phenotype model 122 recovers the original phenotype state. Likewise, the compound-to-phenotype 142 and the phenotype-to-compound 144 models and the omics-to-compound 162 and the compound-to-omics 164 model can be trained to satisfy respective recovery constraints.

In particular, the combination of the models 120, 140, and 160 can create a unified relationship between the omics space 110, the omics space 130, and the compound space 150, which can be leveraged for drug discovery. As an example, the compound-to-omics model 164 and omics-to-phenotype 122 model can be jointly optimized to select promising candidate drug compounds. In this case, the system 100 can link the compound and phenotype spaces using the omics space as a bridge in order to probe the impact of an identified candidate compound in a subject. More specifically, the system can use the compound-to-omics model 164 to predict an omics perturbation, e.g., the change in the omics state, as a result of administering the candidate compound to a subject, and use the omics-to-phenotype 142 model to predict the corresponding phenotype perturbation, e.g., the change in the phenotype state, as a result.

As another example, the system 100 can use the omics-to-phenotype model 122 to associate the omics space 110 with a phenotype space 130 and determine how phenotype states change as a result of perturbations to omics states. In particular, the system can generate data defining a set of paths through the space of phenotypes by perturbing an omics state. For instance, the system 100 can generate a path starting from a “disease” phenotype (e.g., a phenotype state of a person that has a disease) to a “healthy” phenotype (e.g., a phenotype state of a healthy person, e.g., a person that does not have the disease).

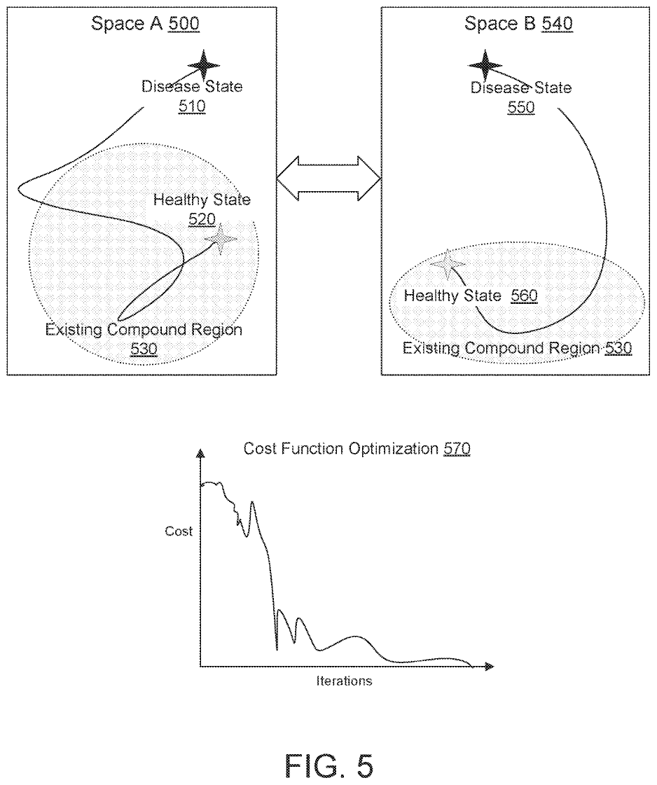

In this context, a path is a sequence of states, e.g., starting from a first state and ending at a final state. In particular, a path can include a sequence of one or more perturbations from a first state to a second state, e.g., each state in the sequence after the first state can be separated from the preceding state by less than a threshold distance. For example, the distance between states can be quantified using one or more distance measures, e.g., a Euclidean distance measure, cosine distance measure, etc., or between numerical representations, e.g., embeddings of the states. More specifically, a “path” through the space of phenotypes can refer to an ordered sequence of phenotypes, and a “path” through the space of omics data can refer to an ordered sequence of omics states. An example of generating a path of phenotype and omics states from a disease state to a healthy state will be described in more detail in FIGS. 5-8.

In particular, the system 100 can be used to characterize which type of perturbation, e.g., a knock out, knock down, knock up of a gene or a combination of genes, drives a certain disease using a path in phenotype space 130, omics space 110, or both. At the cellular level, a disease can be modeled as a combination of phenotypes of interest, e.g., lysosomal function, mitochondrial function, etc. Instead of running a number of successive assays to evaluate phenotype changes from perturbing an identified omics state, e.g., a target gene, the system 100 can use the phenotype-to-omics model 124 to predict corresponding phenotype state changes resulting from updates to omics states.

As yet another example, the system 100 can use the phenotype-to-omics model 124 to associate the phenotype space 130 with an omics space 110, e.g., a genotype space 112 or a proteomics space, and determine how the omics states change as a result of perturbations to phenotypes. Thus, for any appropriate omics data (e.g., genomics data or proteomics data), the system 100 can determine an association between the phenotype space and the omics space.

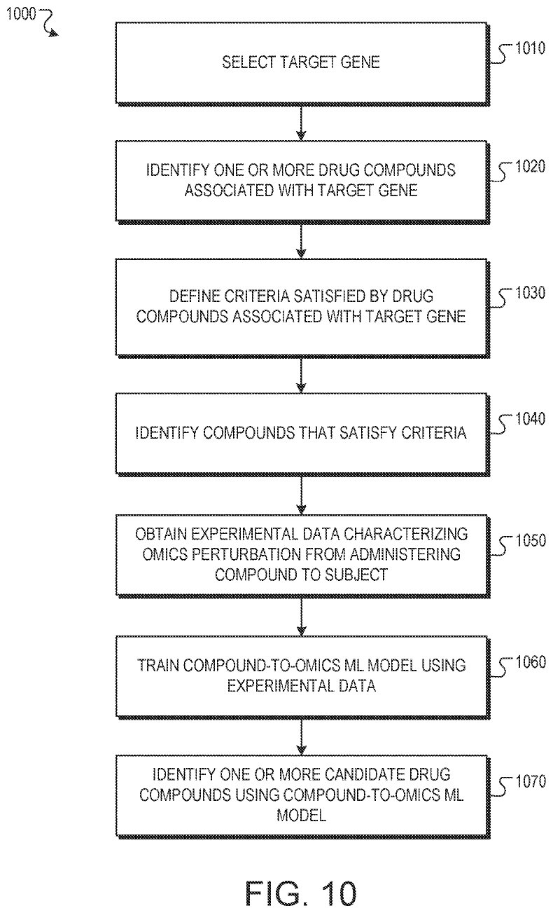

As a further example, the compound-to-omics model 160 can be used to identify one or more candidate drug compounds for a disease. In particular, the system 100 can determine a predicted omics perturbation that would result from administering the candidate drug compound to a subject. An example of identifying one or more candidate drug compounds for a disease using predicted omics perturbations will be described in more detail with respect to FIG. 10.

The system 100 can additionally obtain experimental data 170 from administering the candidate drug compound to one or more subjects that can be used to further train the generative models 120, 140, and 160. In some cases, the experimental data 170 can be derived from cellular assays. In particular, the system 100 can update the compound-to-omics machine learning model 124 and the omics-to-compound machine learning model 122 using the experimental data 170. The experimental data 170 can also inform the training of the compound-to-phenotype 142 and phenotype-to-compound model 144, e.g., based on the impact of the compound on the phenotype expressed by the subject. Furthermore, since the OPC unification system 100 effectively unifies the omics space 110, phenotype space 130, and the compound space 150, the experimental data 170 can inform the training of the omics-to-phenotype 122 and phenotype-to-omics 124 models as well.

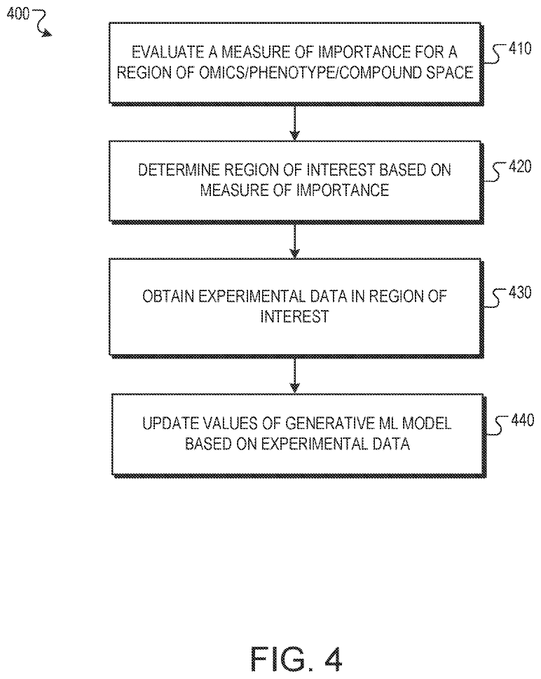

In some cases, the system 100 can identify a need to obtain experimental data 170 in a region of omics 110, phenotype 130, or compound space 150 where the amount of data might be lacking, e.g., using active learning. In particular, the system 100 can evaluate a measure of importance indicative of relevance with respect to a disease and obtain experimental data 170 in accordance with an identified region of high importance. In the case that the omics, phenotype, or compound data space is represented as a biological data graph, the system can incorporate the experimental data 170 into the graph using the graph neural network, e.g., to add a new edge or node to the graph representation in order to update the generative model 120, 140, and 160. An example for using active learning to train the one or more generative models will be covered in more detail in FIG. 4.

FIG. 2 depicts an example biological data graph that includes nodes representing biological entities and edges that connect a respective pair of nodes and represent a relationship between the biological entities corresponding to the respective pair of nodes. As an example, the OPC unification system 100 of FIG. 1 can use the node and edge embeddings of the biological data graph to train the one or more embedding models of FIG. 1.

Biological data graphs integrate data into a common framework that maintains the relationship between nodes in an ontology, which is a representation of the node entities and how they are linked together in the graph. For example, the biological data graph can be described in terms of a topology that represents the relational structure and geometric organization of the graph. In particular, a “graph” can include: (i) a set of nodes, and (ii) a set of edges, where each edge can connect a respective pair of nodes. As an example, each node can represent a respective biological entity and each edge can represent an association or relationship between a respective pair of nodes. For instance, an edge can encode a relationship such as: “is associated with”, or “is caused by”, or “is experimentally observed”, and so forth.

In some cases, the biological data graph 200 can be a heterogenous data graph. In this case, the nodes can be heterogeneous, e.g., the biological entities of the nodes in the graph do not need to be the same type of biological entity. In particular, the biological entity nodes can include genotype nodes, phenotype, and compound nodes, e.g., known drug nodes. As another example, the system can obtain a set of biological data graphs, e.g., each corresponding to a homologous omics graph, phenotype graph, and compound graph.

The relationships represented by edges can include relationships that pertain to the types of biological entities that the edges connect. As an example, the type of biological entity of each node can be maintained as a label, such as a node representing a compound being designated as a compound-type node and a node representing a gene as a gene-type node. In an example in which an edge relates two gene-type nodes, the relationships can include co-expression or coregulation.

In the particular example depicted, both node A 210 and node B 220 are connected by an edge 215. As an example, the edge 215 between node A 210 and node B 220 can represent a relationship between, e.g., a compound of node A 310 that inhibits a gene of node B 330. As another example, other compound-type node to gene-type node edges can include edges representing increased gene expression or modification.

Each node and edge in the graph 200 can be associated with an embedding, e.g., the system can maintain the data representing the nodes and edges as embeddings. In the particular example depicted, embedding 1 212 corresponds with the node 1 210, embedding 2 222 corresponds with the node 220, and embedding 3 230 corresponds with node 3 230.

The system can incorporate additional information, e.g., experimental data, into the biological data graph 200 by processing the new data to generate one or more additional embeddings that can be incorporated into the graph 200 using an embedding model, e.g., the embedding models 105, 135, and 155 in FIG. 1. In particular, the system can use a graph neural network (GNN) as the embedding model to process the current embeddings, e.g., the existing node and edge embeddings, and the additional embeddings to add a new node, edge, or both to the graph 200. As an example, the GNN can leverage the fact that the embeddings for neighboring nodes one-hop or a few hops away, e.g., where a hop refers to an edge connection, should be more similar than embeddings for non-neighboring nodes that are located in different regions of the graph, e.g., several edges away.

In particular, training the embedding model can involve optimizing an objective function to encourage an increase in similarity between node embeddings that are connected by an edge in the biological data graph 200 and a corresponding decrease in similarity between node embeddings that are not connected by an edge in the biological data graph 200. More specifically, the edges of the graph 200 as represented in the node embeddings can be used to quantify how similar nodes are amongst neighboring and non-neighboring nodes. For example, a wealth of experimental information may be available on genes in certain disease areas, and the system can integrate this experimental information into the biological data graph 200. The system can use a graph embedding technique (e.g., based on a graph neural network) to convert each gene node to an embedding, e.g., a vector, in a latent space. The system can then use the latent space to define a similarity measure between gene embeddings, e.g., in the node embedding space 250.

More specifically, the similarity between nodes can be quantified as the distance between node embeddings in the node embedding space 250. In the particular example depicted, embedding 1 for node 1 212 is closer to embedding 2 for node 2 222 in node embedding space 250, and embedding 3 for node 3 240 is farther away from embedding 1 in node embedding space 250. For example, node 1 and node 2 have respective relationships with a common node connection: node 4.

In particular, the system can iteratively process the current and new embeddings to update the node and edge embedding in the biological data graph 200 with message passing, e.g., a transductive learning method for updating node and edge embeddings based on the aggregation of information from nearby node and edge embeddings into messages, to update the corresponding embeddings in order to incorporate the new information into the biological data graph 200. As an example, the system can compare a current node embedding and a new node embedding and use the measure of similarity to generate a similarity score between the first and second nodes. The similarity score can then be used to predict an edge between the first node and the second node in the graph representation.

In some cases, the generative models, e.g., the generative models 120, 140, and 160 can be encoder-decoder models that have been trained using the biological data graph 200 to map between a first node-type embedding space and a second node-type space. In this case, the decoder of the encoder-decoder model can reconstruct the data from a node embedding in the graph representation. As an example, the generative model 120 can be an encoder-decoder model trained to map between an omics node embedding space and a phenotype node embedding space. As another example, the generative model 140 can be an encoder-decoder model trained to map between the phenotype node embedding space and a compound node embedding space. As yet another example, the generative model 160 can be an encoder-decoder model trained to map between the genotype node embedding space and the compound node embedding space.

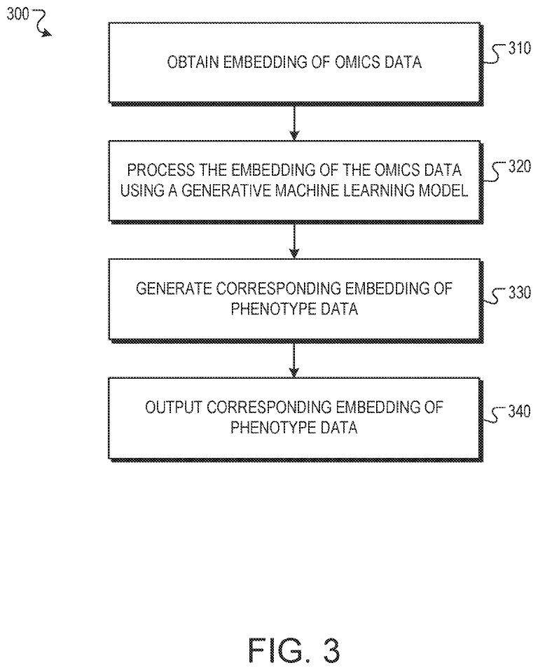

FIG. 3 is a flow diagram of an example process for generating an embedding of biological data using a generative model. While FIG. 3 is directed to an example of processing an embedding of omics data to generate an embedding of phenotype data, the system can perform similar processes to process an embedding of omics, phenotype, or compound data to generate a corresponding embedding of a different biological data type, e.g., corresponding omics, phenotype, or compound data.

For convenience, the process 300 will be described as being performed by a system of one or more computers located in one or more locations. For example, an OPC unification system, e.g., the OPC unification system 100 of FIG. 1, appropriately programmed in accordance with this specification, can perform the process 300 to process one type of biological data, e.g., omics, phenotype, or compound data, to generate a different type of biological data.

In particular, the system can obtain an embedding of omics data (step 310). As an example, the omics data can include one or more of genomic data, or proteomic data, or transcriptomic data, or epigenomic data, or metabolomic data. In some cases, the system can obtain the embedding of the omics data using a graph representation of a collection of omics data, e.g., a biological data graph as described with respect to FIG. 3. For example, the graph representation can include a set of nodes, e.g., where each node in the graph representation is associated with a respective node embedding of each of the one or more omics states, and a set of edges, where each edge connects a respective pair of nodes and represents a relationship between the pair of omics states.

The system can process the embedding of the omics data using a generative machine learning model to generate an embedding of corresponding phenotype data (step 320), and can generate a corresponding embedding of phenotype data (step 330). As an example, the phenotype data can include one or more of morphological phenotype data, or physiological phenotype data, or biochemical phenotype data, or behavioral phenotype data.

In particular, the system can process the embedding of the omics data using an omics-to-phenotype generative machine learning model, e.g., a diffusion model, that is configured to map embeddings of omics data to corresponding embeddings of phenotype data. More specifically, the system can train the generative machine learning model in accordance with a bidirectionality constraint such that the system can ensure a bidirectional mapping between the omics and phenotype space.

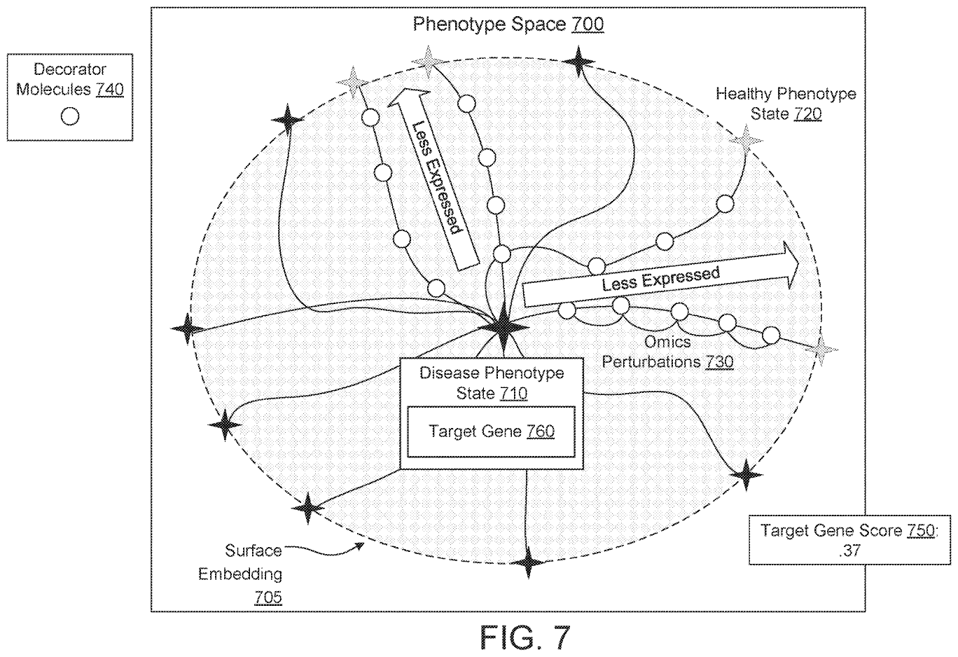

The system can then output the embedding of the phenotype data (step 340), e.g., the system can output the embedding of phenotype data to a user of the OPC unification system 100. For example, the user can use the corresponding embedding of phenotype data to understand how perturbations to omics states can lead to corresponding phenotype changes, as will be described in further detail in FIGS. 7-9.

FIG. 4 is a flow diagram of an example process for training a generative model using active learning. Active learning is an iterative process in which a model is trained on a subset of training data, e.g., labelled data, and then actively selects examples outside of the training set to train on. For convenience, the process 400 will be described as being performed by a system of one or more computers located in one or more locations. For example, an OPC unification system, e.g., the OPC unification system 100 of FIG. 1, appropriately programmed in accordance with this specification, can perform the process 400 to train the generative models 120, 140, and 160.

More specifically, the system can train the generative model on a limited, e.g., limited by the amount of available experimental data, set of training data, e.g., training data that does not characterize every possible combination between the two spaces the generative model maps together, e.g., every possible omics-phenotype, phenotype-compound, and compound-omics combination. For example, training data may be available for only a proper subset of genes, e.g., 1,000 genes in a set of 20,000 genes. The system can incorporate training data from a variety of different sources (e.g., databases) for training the generative model, and then train the generative model using an active learning framework.

In particular, the system can evaluate a measure of importance for a region of omics, phenotype, or compound space (step 410). More specifically, the system can use a corresponding generative model to identify sets of one or more of omics data, phenotype data, compound data, or any combination, for which experimental data is not available and that satisfy one or more additional criteria. In some cases, the system can identify a set of data that is one of the input or output model data type, e.g., the system can evaluate a measure of importance for a region of omics space for the omics-to-compound model. In other cases, the system can identify a set of data that is not the same type as the input or output model data type, e.g., the system can evaluate a measure of importance for a region of phenotype space for the omics-to-compound model.

As an example, the additional criteria can include a relevance criterion, e.g., that the omics data, phenotype, or compound is predicted to be relevant to a particular disease or treatment, e.g., a particular disease or treatment that a user is using the OPC unification system to evaluate. In some cases, the relevance criterion can be determined manually by a physician or using a model that has been trained using sets of phenotypes typical of patients having a particular disease.

As another example, the additional criteria can include a confidence criterion, e.g., a criterion specifying that the generative model has a low confidence in characterizing the omics, phenotype, or compound data in the particular region of the embedding space. In particular, in addition to generating an output omics, phenotype, or compound embedding, the generative model can also generate a confidence measure indicating a confidence in prediction. In the case in which the generative model is implemented using a neural network, the system can generate the confidence score by processing an intermediate output of the neural network, e.g., using an additional output head in a diffusion model implemented using a denoising neural network or a generative adversarial network. In particular, the system can train the additional output head to regress a difference between (i) the predicted output generated by the generative model and (ii) the target output, e.g., to optimize a loss function that measures an error between: (i) the predicted confidence score and (ii) a target confidence score based on the error between the predicted output and the target output.

The system can generate a respective measure of importance for a number of omics, phenotype, and compound states for which experimental data is not available. In some cases, the system can employ random sampling or archetypal analysis to ensure a representative distribution of evaluated states, e.g., either in a model input or output data type space or in a different data type space. For instance, the system can implement an active learning framework that enables the phenotype-to-omics model to leverage available experimental data in particular regions of the genotype and omics spaces, e.g., from available clinical data, in order to learn how to perform the mapping in the whole region of genotype and phenotype space. In particular, active learning can be used to generate data in regions of phenotype, omics, or compound space that is relevant to the disease, e.g., with respect to a target omics state, identified compound, or phenotype that corresponds with a particular symptom of the disease.

The system can then determine a region of interest based on the measure of importance (step 420), e.g., by ranking the evaluated regions according to the measure of importance, and can obtain experimental data in the region of interest (step 430). As an example, the experimental data can characterize the selected omics, phenotype, compound data, or any combination. More specifically, the system can obtain experimental data derived from real-world experimental measurements, or simulations, or a combination thereof to supplement the generative model. In particular, the system can inform a user to perform laboratory experiments, e.g., low-to medium-throughput assays, to measure phenotypes in response to gene perturbations. The system can incorporate such experimental data to extend predictions for genes (or combinations of genes) which have not been experimentally perturbed, e.g., to augment the training of the generative model.

The system can then update the values of the generative machine learning model based on the experimental data (step 440). In particular, the system can incorporate the experimental data into the graph, e.g., using message passing as described in FIG. 2. The generative machine learning model can then be trained using the updated graph, e.g., using a machine learning technique, e.g., by performing gradient descent using a set of training examples. For example, the system can employ techniques as demonstrated in “High-Resolution Image Synthesis with Latent Diffusion Models” (Rombach, R., et. al. Proceedings of the IEEE/CVF conference on computer vision and pattern recognition. 2022) or “GRAND: Graph Neural Diffusion” (Chamberlain, B., et. al. PMLR 131 2021) to train the generative machine learning model. In particular, training with the updated graph can strengthen the predictive capabilities of the generative model in the particular region of interest identified.

In particular, the system can leverage the one or more biological data graphs, e.g., the biological data graph 200 of FIG. 2, to infer the behavior of omics states, phenotypes, or compounds that are not included in the one or more biological data graphs. More specifically, the system can chart the impacts of perturbing a state in one space, e.g., an omics space, and evaluate the impacts in a second space, e.g., a phenotype space. Inferring combinations of states not included in the biological data graph can allow for the discovery of candidate compounds that were unlikely to be discovered otherwise, e.g., through a literature search. For example, the system can use a homogeneous omics graph including gene data, e.g., a graph in which each node corresponds to a gene and each node in the graph is associated with a gene embedding, to train the omics-to-phenotype and omics-to-compound models, e.g., the models 122 and 162, and update the graph using active learning.

In some cases, the system can also use the information included in the biological data graph to infer the behavior of a biological entity, e.g., a gene, phenotype, or compound, for which data is not available. In particular, the system can evaluate “closeness” between biological entities, e.g., by comparing embeddings representing the entities in a latent embedding space using an appropriate similarity measure, e.g., a similarity measure based on an inner product between embeddings. The system can then use the closeness of the biological entity for which data is not available to a biological entity for which data is available to infer corresponding behavior. As an example, the system can use the closeness of a gene for which data is not available to a gene for which data is available to infer the similarity of their phenotypic impacts, e.g., the “similar” genes will produce similar perturbations.

The system can repeat the process 400 at each of a sequence of training iterations, e.g., “active learning iterations”. In each active learning iteration, the system can obtain new experimental data and further train the generative model on the new experimental data, e.g., such that predictions can improve in the identified region of interest. In particular, the importance scores can become more accurate as a result of the active learning at each active learning iteration, e.g., such that the model can specialize in learning how to generate embeddings within the most relevant regions of omics, phenotype, and compound data for a particular disease.

FIG. 5 illustrates how the system can identify an example path from a disease state to a healthy state in a first space, e.g., a phenotype space, and generate the corresponding space in a second space, e.g., an omics space. In this case, a “path” through the space of phenotypes can refer to an ordered sequence of phenotypes, and a “path” through the space of omics data can refer to an ordered sequence of omics states.

As an example, the system can identify a transition from a disease-type state to a non-disease-type state using the phenotype-to-omics model. More specifically, the system can receive data identifying a set of one or more disease phenotypes associated with a disease and a set of one or more healthy phenotypes that are not associated with the disease and can determine paths that each start from a respective disease phenotype and terminate at a respective healthy phenotype. As an example, the data identifying the set of one or more disease phenotypes can be specified by an expert or generated by an automated upstream system, e.g., by a system configured to mine electronic medical records.

In the particular example depicted, the system can generate data defining a path through a first space, e.g., the space A 500. As an example, the space A 500 can correspond with the space of phenotypes. For instance, the system can generate a path starting from a “disease” phenotype, e.g., a phenotype state 510 of a person that has a disease, to a “healthy” phenotype, e.g., a phenotype state 520 of a healthy person, e.g., a person that does not have the disease. In particular, the system can successively perturb the phenotype disease state 510 until a healthy state 520 is reached.

As an example, perturbing the phenotype state can involve updating the embedding of phenotype data according to the desired perturbation. More specifically, the system can determine paths of intermediate phenotypes in the sequence of phenotypes by interpolating between a higher-dimensional point associated with the disease phenotype state and a point associated with the healthy phenotype state, e.g., using linear interpolation or by optimizing a path according to a cost function, as will be described in further detail below.

The system can then process the path in the space A to generate a corresponding path in space B 540, e.g., by sequentially processing each individual state in the path using the generative model to generate a corresponding output state such that the output path is the sequence of output states. As an example, the system can use the phenotype-to-omics model, e.g., the phenotype-to-omics model 124, to process the data defining the path in phenotype space to generate data defining a corresponding path in omics space. In particular, the system can map the path through phenotype space into a corresponding path through the omics space, e.g., from the disease state 550 to the healthy state 560, e.g., using the phenotype embedding model. For example, the system can identify specific phenotypes, e.g., the disease phenotype 510 or healthy phenotype state 520 not associated with a disease, and process the embeddings of the phenotype states to generate the corresponding omics states. As another example, the system can generate data defining the path in an omics space, e.g., the space A 500 can be an omics space, and the system can further map the path through the space of embeddings into a corresponding path through the phenotype space, e.g., using the omics-to-phenotype model 122.

The system can use the set of phenotype embeddings and corresponding omics embeddings to identify an omics state that precipitates a transition from a disease-type state to a non-disease-type phenotype state. In particular, generating a path from a diseased phenotype state to a healthy phenotype state, and then identifying a corresponding path in the omics (embedding) space, can enable the system to identity changes in the omics state of a subject, e.g., in the genomic, or proteomic, or metabolomic state of the subject, that are associated with transitioning the patient to a healthy state. Thus, the path through the omics (embedding) space can inform the discovery of treatments (e.g., drugs) for transitioning the omics state of a subject from a state corresponding to a disease phenotype to a state corresponding to a healthy phenotype.

In some implementations, the system can generate paths through the first space, e.g., the space A 500, in a manner that is informed by constraints on the corresponding paths in the second space B 540. In the particular example discussed above, the system can generate paths through the phenotype space in a manner that is informed by constraints on corresponding paths traced out through the omics space.

In particular, the system can implement a cost function that can associate any path in the space A 500 or space B 540 with a corresponding cost, e.g., that characterizes a desirability or utility of the path through the space using a numerical value. As an example, the system can perform a cost function optimization 570 over a number of iterations to identify paths through the space A 500, e.g., the phenotype space, that are associated with the lowest possible costs in the space B 540, e.g., the omics space, as measured using the cost function.

As an example, the system can require that paths traced through the omics space avoid certain regions of the omics space, e.g., regions that are non-druggable or that are associated with certain diseases. In this case, non-druggable regions can correspond with regions of compound space that do not have associated compounds. In particular, the system can implement a cost function that encourages the selection of paths through omics space that are associated with existing compounds 530, e.g., a region that can be different between space A 500 and space B 540. In this manner, the system can prioritize the generation of feasible paths through phenotype space (and thus, paths through omics space) that can be associated with existing drugs or molecules, thus facilitating drug discovery and drug repurposing.

As another example, the system can generate paths through the omics space in a manner that is informed by constraints on corresponding paths traced out through the phenotype space. For instance, the system can require that paths traced through the phenotype space avoid certain regions of the phenotype space, e.g., regions that are associated with adverse symptoms (e.g., pain, nausea, depression, etc.) or certain diseases. As another example, the system can randomly generate paths connecting the initial state to the terminal state.

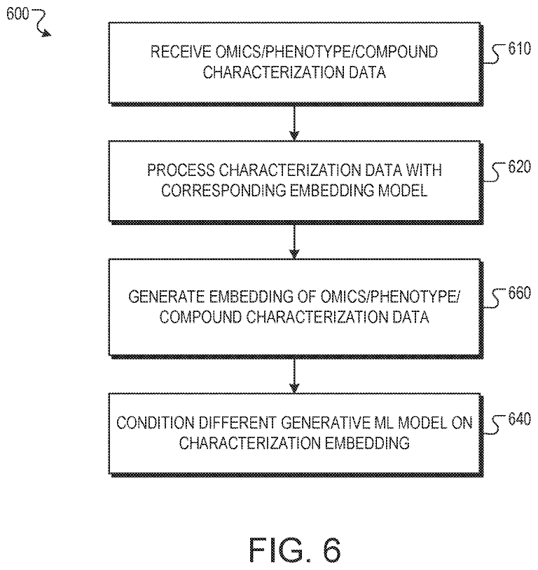

FIG. 6 is a flow diagram of an example process for conditioning different generative machine learning models on characterization embeddings, e.g., characterization embeddings of another biological entity data type. More specifically, conditioning the generative machine learning models refers to modifying the models, e.g., by using the characterization embeddings to modify the output of the model. For convenience, the process 600 will be described as being performed by a system of one or more computers located in one or more locations. For example, an OPC unification system, e.g., the OPC unification system 100 of FIG. 1, appropriately programmed in accordance with this specification, can perform the process 600.

In particular, the system can receive characterization data (610), e.g., omics, phenotype, or compound characterization data. The characterization data can be used to modify the output of a generative model, e.g., in order to inform the perturbation of a biological entity state with respect to the characterization data. As an example, the system can receive data characterizing a compound for use in conditioning a phenotype-to-omics or omics-to-phenotype model. The system can then process the characterization data with the corresponding embedding model (step 620) to generate the embedding of omics, phenotype, or compound characterization data (step 630).

The system can then condition a different generative machine learning model using the characterization embedding (step 640), e.g., the system can use a phenotype characterization embedding to condition a compound-to-omics model. In particular, the system can process the characterization embedding in accordance with the values of the set of generative machine learning model parameters and the characterization embedding to generate a conditioned output. As an example, the characterization embeddings can be used as an additional input to the generative model. As yet another example, the system can incorporate the characterization embeddings into the generative model architecture, e.g., the embeddings can be used to configure one or more of the initial layers of the generative model.

In particular, the system can provide compound embeddings generated by the compound embedding model for use in conditioning the omics-to-phenotype or phenotype-to-omics model, e.g., the system can use the compound embeddings as characterization embeddings to condition the generative neural network. In this case, the conditioned output can be an embedding of corresponding phenotype data indicative of a particular phenotype predicted to arise from the compound being administered to a subject. More specifically, the system can use the characterization embeddings to inform the perturbation of biological entity states, e.g., to inform optimization of a cost function in a different space.

FIG. 7 illustrates an example of analyzing generated paths. In particular, the system can use the identified paths from disease states to healthy states to inform drug discovery, e.g., by identifying a change in gene level expression along a path or identifying one or more decorator molecules associated with corresponding omics states along the path.

In particular, the system can identify a transition from a disease-type state to a non-disease-type state by generating paths in the embedding space of a biological entity, e.g., the omics, phenotype, or compound space. More specifically, the system can receive data identifying a set of one or more disease phenotypes associated with a disease and a set of one or more healthy phenotypes that are not associated with the disease and can determine paths that each start from a respective disease phenotype and terminate at a respective healthy phenotype, e.g., using a cost function optimization.

In some cases, the system can define multiple paths, e.g., by performing different first perturbations to the starting disease state and propagating along the multiple paths defined by the different first perturbations. For example, the system can define multiple paths in phenotype space from a first phenotype state, e.g., the disease phenotype state, or multiple paths in omics space from a first omics state. In this case, the multiple paths can be combined into a surface embedding, e.g., an embedding that combines the data defined by the multiple paths, in either phenotype or omics space, respectively.

In the particular example depicted, the system has defined multiple paths in a phenotype space 700 starting from a disease state 710. In this case, the multiple paths can be combined into the surface embedding 705 in the phenotype space 700. More specifically, the system can define multiple paths characterizing multiple sequences of phenotype states from the disease phenotype state to the healthy phenotype state 720, which can be combined into a surface embedding 705 in phenotype space.

In particular, the system can define paths in phenotype space that cover a strategic region of omics space, e.g., the system can define a path from a disease state by strategically perturbing the phenotype to result in different paths that can span different directions and regions in omics space, thereby generating the surface embedding 705. Likewise, the system can also define paths in omics space that cover a strategic region of phenotype space. More specifically, the system can control the span of the surfaces generated in phenotype space and omics space by strategically applying successive perturbations.

The system can then process the surface embedding 705 in phenotype space 700 to generate a corresponding surface embedding in omics space. In this case, the omics embedding characterizes differential gene expression for the omics state that corresponds with the disease phenotype state 710. More specifically, the system can process each path in phenotype space to generate a corresponding path in omics space, e.g., using the phenotype-to-omics machine learning model 124. The corresponding paths in omics space can then be used to identify one or more target omics states, e.g., the target gene 760, which is associated with the disease phenotype state 710.

In particular, in the case that the majority of phenotype states on the phenotype surface 705 are non-disease type phenotypes, the omics state corresponding to the disease phenotype 710 can be considered a target omics state. In the case that a few of the associated phenotype states are non-disease-type, e.g., most remain as disease-type phenotype states, then the omics state can be considered a correlative omics state. In particular, the omics state might be part of a downstream pathway or a distal response, e.g., inflammation.

The identification of target omics states can inform the identification and administering of a drug that targets the target omics state. In particular, generating one or more paths from a disease phenotype state to a healthy phenotype state, and then identifying one or more corresponding paths in the omics space, can enable the system to identity a link between changes in the phenotype state of a subject and omics states that are associated with transitioning the patient to a healthy state, e.g., using the corresponding omics surface.

Each of the omics states along the omics path can correspond with a sequence of one or more perturbations applied to the first omics state, e.g., the target gene 760. In particular, each sequence of phenotype perturbations in each path can correspond to a sequence of omics perturbations, e.g., the omics perturbations 730. In particular, the system can process the data defining the omics paths to identify a set of target omics states, e.g., omics states that are predicted to be causative with respect to phenotype states associated with the disease. For example, expression of the target omics state, e.g., the target gene 760, can be predicted to either activate disease pathway or inhibit or modify a healthy pathway to cause a disease pathway. In the case that the omics state is a gene, the system can identify target genes based on a level of variation of expression of the gene. For example, the system can identify one or more genes associated with the highest variance of expression as the target gene 760.

As an example, the omics state perturbations corresponding with the phenotype perturbations can include activating or inhibiting the preceding omics states, e.g., using one or more compounds that target the preceding omics state in the sequence. In particular, respective perturbations can result in a differential expression of the one or more genes in the omics state, e.g., a knock-out, down, or up perturbation of the omics state. In the particular example depicted, the omics perturbations leading to healthy phenotype states, e.g., the state 720 correspond directly with decreased expression of one or more omics states, e.g., genes, associated with the disease, e.g., as represented by the arrows showing the direction of comparably less gene expression with respect to the target gene 760. In this case, the healthy phenotype state 720 correlates with a lower level of expression of the target gene 760.

The sequence of omics states defined by the path in omics space can additionally correspond with a number of molecules, e.g., target omics states or correlative omics states, e.g., biomarkers. In particular, the omics paths can be “decorated” by one or more decorator molecules 740 that are associated with the omics states in the omics sequence defined by the omics path. In this case, the decorator molecules can be candidate biomarkers.

As an example, the system can identify one or more biomarkers that correlate with the target omics state, e.g., the target gene 760, as correlative omics states. In particular, biomarkers can be produced by the subject or move to different locations in the subject in response to the omics change, e.g., due to circulation. For example, central nervous system fluid and proteomic fluid can include one or more biomarkers as a result of an omics state perturbation. The system can identify correlative omics states that can be analyzed to provide more information about the disease associated with the disease phenotype state and inform the drug discovery process.

After generating the phenotype surface embedding 760 and evaluating the corresponding omics embedding, the system can identify if there is a transition from a disease-type state to a non-disease type state on the phenotype boundary of the surface for each of the determined target omics states. More specifically, the system can determine a ranking of target omics states using the corresponding phenotype surface embedding 760.

In some cases, the system can determine a classification for each path associated with a given target omics state by specifying whether the omics path terminates at a healthy phenotype, and determine a score, e.g., the target gene score 750, based on the path classifications for the target omics state. In particular, the system can determine a respective score for each of the target omics states, e.g., using an aggregation of a classification of healthy and unhealthy paths or by predicting a value for each of the omics paths based on a measure of a likelihood that the omics states along the path are expressed in the healthy state.

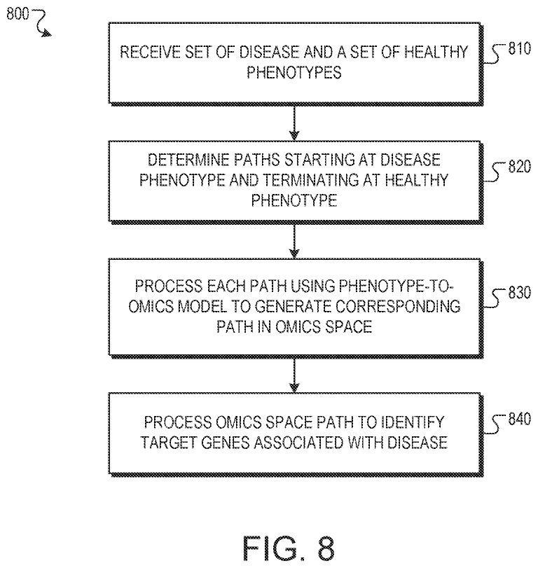

As an example, the system can calculate a fraction of the healthy paths, e.g., the paths starting from a target omics state and terminating at a healthy phenotype, as the score for the target gene 760. More specifically, the system can compare the number of disease-type or non-disease type classifications for the phenotype states associated with the boundary of the phenotype surface 760 that corresponds with the boundary of the omics surface corresponding to the target omics state to determine a fraction of healthy paths, e.g., four out of 11 paths. The system can then use the scores to rank the target omics states, e.g., as a proxy for determining causality between the target omics state and the corresponding related healthy or disease phenotypes, as will be described in further detail with respect to FIG. 8.

FIG. 8 is a flow diagram of an example process for identifying target genes associated with a disease using paths in phenotype and omics space. In particular, FIG. 8 further describes how the system can use the identified paths from disease states to healthy states to inform drug discovery. For convenience, the process 800 will be described as being performed by a system of one or more computers located in one or more locations. For example, an OPC unification system, e.g., the OPC unification system 100 of FIG. 1, appropriately programmed in accordance with this specification, can perform the process 800.

In particular, the system can receive a set of disease and a set of healthy phenotypes (step 810), and the system can determine paths starting at the disease phenotype and terminating at a corresponding healthy phenotype (step 820). For example, the system can define sequences of perturbations as paths that lead from the disease state to the healthy phenotype, as described in FIG. 8.

As an example, in the case of a disease phenotype state that is associated with the overexpression of five genes, the system can define a path in phenotype space that corresponds with independently reducing the overexpression of the five genes, e.g., by identifying the paths that result from using independent sequences of successive perturbations. In particular, the system can optimize the phenotype paths based on the corresponding path in omics space, e.g., for each omics state, the system can strategically apply sequences of successive perturbations to span a region around each of the omics states, e.g., each overexpressed gene.

The system can then process each path using a phenotype-to-omics model to generate a corresponding path in omics space (step 830). More specifically, the system can map the phenotype path into the omics embedding space. Generating a path from a diseased phenotype state to a healthy phenotype state, and then identifying a corresponding path in the omics space, can enable the system to identity relevant changes in the omics state of a subject, e.g., in the genomic, or proteomic, or metabolomic state of the subject, that are associated with transitioning the patient to a healthy state.

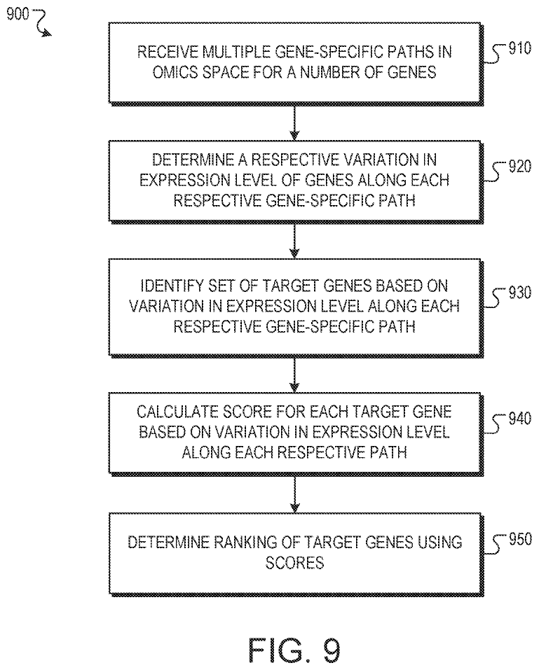

The system can then process the omics space path to identify target genes associated with the disease (step 840). In particular, the system can identify and rank genes that produce desirable perturbations in relevant phenotypes as target omics states, as will be described in further detail with respect to FIG. 9.

FIG. 9 is a flow diagram of an example process for ranking target genes using the corresponding paths in phenotype and omics space. For convenience, the process 900 will be described as being performed by a system of one or more computers located in one or more locations. For example, an OPC unification system, e.g., the OPC unification system 100 of FIG. 1, appropriately programmed in accordance with this specification, can perform the process 900, e.g., as part of step 840 in FIG. 8.

The system can receive multiple gene-specific paths for a number of genes (step 910), e.g., from the system determining paths starting at a disease phenotype and terminating at a healthy phenotype in step 920 of FIG. 9. The system can determine a respective variation in expression level of the genes along each respective path (step 920). In particular, the system can use the phenotype-to-omics model to process each phenotype in the sequence of phenotypes in the gene-specific path from the disease phenotype to the healthy phenotype to generate a corresponding omics state and can quantify the variation in the expression level of the gene along the sequence of corresponding omics states. For example, the system can quantify the variation as the difference between the maximum expression and minimum expression of the gene along the path.

The system can then identify a set of target genes based on the variation in expression level along each respective gene-specific path (step 930). More specifically, the system can identify one or more target genes associated with the highest variation in expression level, e.g., the system can identify the top N % genes with the highest variation of expression as target genes or the genes with variation level above a certain threshold as target genes. For example, target genes can be genes with paths that start at a high level of expression and terminate at a low level of expression, e.g., oncogenes, e.g., genes overexpressed in various cancers, tumor suppressor genes, genes involved in neurodegenerative diseases or genetic disorders, etc. As another example, target genes can be genes with paths that start a low level of expression and terminate at a high level of expression, e.g., tumor suppressor genes, genes involved in DNA repair, apoptosis-related genes, an insulin gene, neurotrophic factors, telomerase, etc.

The system can then calculate a score for each target gene based on the variation in expression level along each respective gene-specific path associated with each target gene (step 940). In particular, the system can determine, for each of the target genes, a ranking based on the multiple gene-specific paths associated with each target gene. As an example, the system can determine a respective score for each target gene, e.g., by determining a path classification for each gene-specific path that indicates whether the gene-specific path terminates at a healthy phenotype and aggregating the path classifications as the score. In particular, the system can determine the score for each target gene based on a fraction of gene-specific paths for the target gene that are classified as terminating at a healthy phenotype. The system can then determine a ranking of target genes using the scores (step 950), e.g., by ranking the target genes in descending order based on the determined score.

FIG. 10 is a flow diagram of an example process for identifying candidate drug compounds for an identified target gene associated with a particular disease. For convenience, the process 1000 will be described as being performed by a system of one or more computers located in one or more locations. For example, an OPC unification system, e.g., the OPC unification system 100 of FIG. 1, appropriately programmed in accordance with this specification, can perform the process 1000.

The identification and ranking of target omics states, e.g., as described in process 1000 in FIG. 10, can additionally inform the characterization of drug compounds that are associated with treating the disease. More specifically, the system can assess drug compounds associated with the target omics states to determine a set of one or more properties that characterize the drug compounds, e.g., similar properties of the identified drug compounds. The system can then use the properties to identify candidate drugs that target the set of one or more target genes and determine that a patient having the disease should receive the identified drug.

In particular, the system can select a target gene that is predicted to be associated with a disease (step 1010). In particular, the target gene can have been identified using the process 900 detailed in FIG. 9. As an example, the system can have generated multiple phenotype surface embeddings from a phenotype disease state and can choose the target gene corresponding with the disease phenotype state with the highest score, e.g., the target gene associated with the greatest number of paths terminating at healthy phenotype states.

The system can then identify one or more drug compounds associated with the target gene (step 1020) and can define criteria satisfied by the drug compounds associated with the target gene (step 1030). More specifically, the system can determine a set of one or more properties that are predicted to be satisfied by drug compounds that act on the target gene using the one or more drug compounds that are associated with a target gene. As an example, the set of one or more properties can include a binding affinity, e.g., for the compound with a protein corresponding with the expressed target gene.

As another example, determining the set of one or more criteria can include performing a quantitative structure-activity analysis (QSAR) to the identified drug compounds. In particular, performing QSAR can involve processing the chemical structure of the identified compounds using a QSAR model and generating the physicochemical, biological, and environmental fate properties of each identified drug compound from the chemical structure. As an example, in the case that one of the identified drug compounds is a protein, the QSAR can characterize the hydrophobic and lipophilic portions of the protein in order to provide an indication of binding activity.

The system can then identify compounds that satisfy the criteria (step 1040). In particular, the system can sample compounds from a space of compounds that satisfy the set of one or more criteria, e.g., in accordance with importance sampling determined by the set of one or more criteria. As an example, the system can sample over the set of molecules that satisfy the criteria using a uniform distribution. In some cases, the system can apply one or more constraints to the compound space based on the physiochemical, biological, and environmental fate properties derived from the chemical structure. In particular, the system can apply one or more constraints according to criteria deemed relevant to the compound space with respect to the target omics state.

As an example, the size of the compound, manufacturability, ADME (absorption, distribution, metabolism, and excretion) profile, toxicity, polar surface area, and mutagenesis constraints can be applied to restrict the space of compounds for sampling. As another example, Lipinski's rule for pharmacologically active substances, e.g., that a compound can have no more than five hydrogen bond donors, no more than 10 hydrogen bond acceptors, a molecular mass less than 500 Daltons, a calculated octanol-water partition coefficient of five, etc. can be used to constrain the compound space. As yet another example, a docking constraint can be applied to assess feasibility of a compound docking with the target protein molecule. In particular, the system can predict the binding affinity, e.g., the binding activity of the compound by calculating a flexible or rigid ligand-target docking score.

In some cases, the system can apply one or more modifications to a respective one or more of the identified drug compounds to generate synthetic data, e.g., modified compounds within a similar region of compound space. In particular, the modifications can include one or more of translations, rotations, or torsions of the molecular structure compound. In particular, applying modifications can create ligand-pose transformation data, e.g., with respect to different potential docking configurations of a compound, that can augment the experimental data. In some cases, modifying the molecular structure of the compound can result in a different effect on the target omics state than the identified drug compound.

In some cases, the system can additionally analyze the compounds that resulted from the modifications to the identified drug compounds and filter the compounds identified based on the properties identified, e.g., size, weight, manufacturability, toxicity, solubility, topological polar surface area, docking, etc. In some cases, this filtering is done to identify an important region of compound space. As an example, the set of criteria can include ranges for one or more properties, e.g., a molecular weight between 100-500 g/mol, a solubility between 8-20 mg/mL, etc.

The system can then obtain experimental data characterizing the omics perturbation from administering the drug compound to subject (step 1050), e.g., the difference between respective numerical representations of the omics state of the subject before and after receiving the compound. In particular, the system can identify a number of drug compounds that satisfy some or all of the set of criteria and obtain experimental data that characterizes omics perturbations based on results from administering the drug compounds identified to a subject. As an example, this can involve performing one or more assays to assess omics perturbations, e.g., Reverse Transcription Polymerase Chain Reaction, Quantitative Real-Time PCR, Northern Blotting, RNA Sequencing, Microarrays, Western Blotting, Reporter Arrays, etc., and the obtained data can be used to fill in a region of omics space, e.g., a region of unknown omics states.

The system can then train the compound-to-omics machine learning model using the experimental data (step 1060), e.g., by creating training examples including a training input and a target output. More specifically, the compound-to-omics machine learning model can be configured to process a compound as training input to generate a predicted omics perturbation as output that can result from administering the candidate drug compound to a subject, e.g., the compound-to-omics machine learning model can generate the predicted outcome of an assay.

In particular, the system can use machine learning to optimize an objective function that measures an error between (i) the output generated, and (ii) the target output. For example, the training examples can include experimental data from the identified compounds, synthetic ligand-pose transformation data as a result of the modifications, or both. In some cases, the compound-to-omics model and omics-to-phenotype model can be jointly trained. In particular, the compound-to-omics model and omics-to-phenotype model can be jointly trained on a discrepancy between identified candidate compounds and experimental data for the identified candidate compounds, e.g., cellular assays that demonstrate the compound's actual impact in omics or phenotype space. In some cases, a reinforcement learning technique can be used, e.g., the compound-to-omics model and the omics-to-phenotype model can be jointly trained on rewards in accordance with selecting candidate compounds that fulfill a set of criteria in omics and phenotype space, respectively.

The system can then use the trained compound-to-omics machine learning model to identify one or more candidate drug compounds (step 1070). For example, the system can generate one or more candidate drug compounds and determine the corresponding predicted omics perturbation by performing an automated search through the space of compounds in order to find compounds that optimize an objective function, e.g., that measure how much the compound increases or decreases the expression of a gene in a subject. In particular, the search can be a grid search, a search performed using a black-box optimization, using a reinforcement learning technique, etc.

As an example of process 1000, the trained compound-to-omics model and omics-to-phenotype model can be used to perform a hit-to-lead campaign for a disease. In particular, the system can identify a disease phenotype, and create one or more sequences of phenotype states between the disease phenotype to a healthy phenotype. The sequences can be combined into a phenotype surface that can be translated into an omics space using a phenotype-to-omics model, e.g., the bidirectional omics-to-phenotype model. The system can then use the corresponding omics surface to identify one or more target omics states, which can be used to identify compounds that are associated with the target omics states, e.g., either through inhibition or activation of target genes. The system can then analyze the identified compounds to determine a set of criteria that characterize similarities between the compounds. In particular, the system can use these similarities as constraints on what properties a compound associated with the target omics state can have and leverage the constraints to limit the region of compound space applicable to the disease. The system can further inform the region of compound space that the compound-to-omics model can sample from, e.g., by performing docking simulations for a set of compounds in the region or physical experiments.

For example, the system can provide a practical dimensionality constraint by defining criteria satisfied by the drug compounds associated with a target omics state. The criteria can restrict the universe of potential compounds for a disease to a subset of feasible compounds by applying constraints to the compound space, e.g., based on the QSAR of the compounds, before sampling compounds to train the compound-to-omics model.

Applying the constraints can create a stable diffusion bridge between compound and omics space in a region of high importance, e.g., a region that can include feasible compounds for the disease. In particular, the system can leverage constraints on what properties the compound should have based on an analysis of compounds associated with target omics states, to generate candidate drug compounds that can be further analyzed in omics space and phenotype space. More specifically, the omics states predicted as a result of administering the compound to a subject and the resultant related phenotypes can be analyzed and a value can be assigned to the omics state and the phenotype state, e.g., to penalize unwanted perturbations or predicted undesirable symptoms from administering the drug. By jointly optimizing the compound-to-omics and omics-to-phenotype models, the system can identify promising candidate drugs that are not predicted to have confounding effects in either omics space, phenotype space, or both.

In some cases, one or more of the identified candidate drug compounds can be synthesized, validated, and administered to a subject with the disease. In particular, physical experiments can be performed, e.g., to test one or more properties of the candidate compound. As an example, physical experiments can be performed to evaluate the absorption, distribution, metabolism, and excretion of the candidate drug in a subject. The system can determine that a subject should receive a candidate drug compound identified using the compound-to-omics machine learning model, e.g., in order to treat a particular condition or disease.

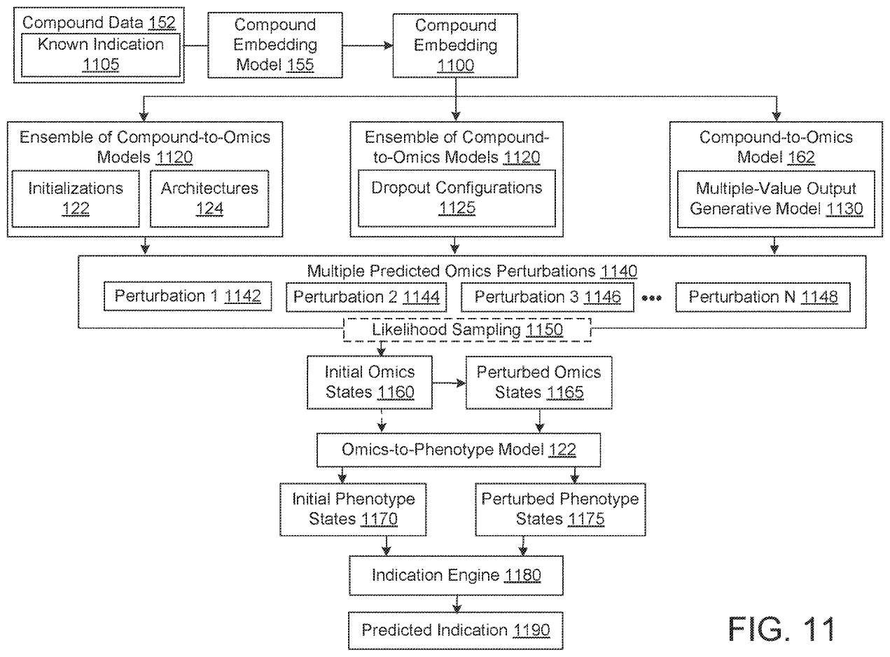

FIG. 11 is a block diagram that illustrates an example of how the system can be used to predict new indications of a drug compound for drug repurposing.

In some cases, a drug can be repurposed, e.g., used for a different purpose than the original disease it was developed and approved for. As an example, aspirin, a drug that is commonly used for pain relief and fever reduction, has been found to be effective at preventing heart attacks and strokes. As another example, bupropion, a drug that is commonly used as an antidepressant, has been found to be effective at having an effect on smoking cessation. As yet another example, sildenafil (“Viagra”), a drug that is commonly used for treatment of hypertension, has been found to treat erectile dysfunction.

In particular, an indication refers to a particular disease that the compound can be used to treat. Drug repurposing involves identifying new indications for existing drugs. For example, metformin, a drug that is commonly used to treat type II diabetes, has been investigated for its potential to treat polycystic ovary syndrome (PCOS). As another example, aprepitant, a drug that is commonly used to treat chemotherapy symptoms, has been investigated for its potential to treat alcohol dependence.

More specifically, the system can receive compound data 152 including a known indication 1105 that can be processed with the compound embedding model 155 to generate a compound embedding 1100. The system can then process the compound embedding 1100 using the compound-to-omics machine learning model 162. In particular, the system can configure the compound-to-omics machine learning model 162 to generate multiple omics perturbation outputs 1140, e.g., the perturbations 1142, 1144, 1146, and 1148, by leveraging a measure of uncertainty in the prediction or by using multiple models to predict respective omics perturbation outputs. In this case, each omics perturbation represents the change in the omics state as a result of administering the drug compound to a subject.

In some cases, the system can use an ensemble of compound-to-omics models 1120, e.g., a set of compound-to-omics models 1120 trained separately. As an example, one or more of the models in the ensemble can have been initialized using a different initialization technique 122 before training, e.g., zero, random, Glorot, He, LeCun initialization, etc., or can be of a different architecture 124, e.g., a diffusion model, a variational autoencoder, or a generative adversarial neural network, or a normalizing flow.

As another example, the system can use dropout regularization, e.g., by inserting a dropout layer, e.g., a layer that randomly removes a subset of intermediate inputs involved in the calculation of the output, e.g., neurons, layers, residual blocks, etc., based on a predefined dropout rate, e.g., a predefined percentage of dropped intermediate inputs, into the compound-to-omics machine learning model 162 during training. In particular, the system can predict multiple omics perturbation outputs using an ensemble 1120 of multiple compound-to-omics models trained with different drop-out configuration 1125, e.g., dropout rates.

In other cases, the system can implement the compound-to-omics machine learning model 162 without an ensemble, e.g., as a single neural network architecture. In particular, the system can implement a generative model that is configured to leverage the uncertainty inherent in the data to output multiple predictions 1130. In particular, the compound-to-omics machine learning model 162 can be configured to generate multiple samples from a distribution over a space of possible omics perturbations. As an example, the compound-to-omics model 162 can be implemented as a Bayesian generative model, Bayesian generative adversarial network, or a variational autoencoder, which can be configured to output a probability distribution, e.g., a range of predictions instead of a single point estimate, e.g., by sampling from a posterior distribution generated using variational inference or Markov Chain Monte Carlo. This range of predictions can then be used to sample multiple omics perturbations 1140.

Furthermore, the system can determine a likelihood of the predicted omics perturbation, e.g., by processing an intermediate output of the one or more compound-to-omics machine learning models 1120 to output a corresponding likelihood of the predicted omics perturbation, e.g., a probability that the predicted omics perturbation can occur given the drug compound was administered to a subject. For example, the system can calculate the likelihood using an additional output head in a diffusion model implemented using a denoising neural network or a generative adversarial network.

In the case that the system determines a likelihood, the likelihoods can be used for likelihood sampling 1150 of the multiple omics perturbations 1140. As an example, each of the multiple omics perturbations 1140 can be associated with a corresponding likelihood score. In particular, the system can sample from the omics perturbations 1140 based on the corresponding likelihood score, e.g., the system can select the top N omics perturbations, as the set of omics perturbations for further evaluation. As another example, the system can randomly sample the set of omics perturbations from a subset of the omics perturbations with a corresponding likelihood greater than a threshold.

The system can then apply the determined set of omics perturbations to a set of initial omics states 1160, e.g., to generate the perturbed omics states 1165. As an example, the system can receive the set of initial omics states. As another example, the set of initial omics states can be identified as a representative set of omics states, e.g., selected in phenotype space as representative phenotype states that a user of the system is interested in probing relative to a particular drug compound. In this case, the system can be used to process each selected phenotype state, e.g., user-selected phenotype state, using a phenotype-to-omics machine learning model, e.g., the phenotype-to-omics model 124, to generate corresponding omics states as the set of initial omics states.

For example, the system can apply an embedding that characterizes each predicted omics perturbation to an embedding that characterizes the initial omics state. In particular, the system can combine, e.g., add, each predicted omics perturbation embedding to each initial omics state embedding. More specifically, the system can generate an embedding of a perturbed omics state as a linear combination of arrays of data characterizing each initial omics state and each omics perturbation.

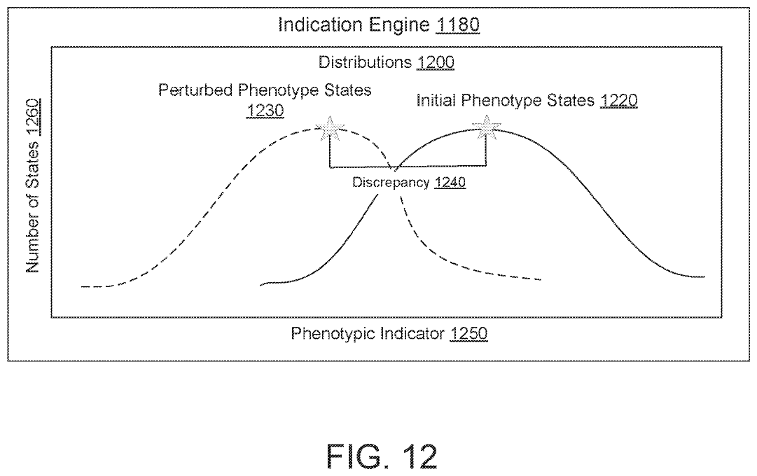

The system can then generate corresponding phenotype states for each of the initial omics state 1160 and perturbed omics state 1165 pairs, e.g., using the omics-to-phenotype model 122. In particular, the system can process the initial omics states 1160 using the omics-to-phenotype model 122 to generate the initial phenotype states 1170 and can similarly process the perturbed omics states 1165 to generate the perturbed phenotype states 1175. As an example, each predicted perturbed phenotype state 1175 can be a predicted outcome of an assay performed to determine a response from administering the drug compound to a subject. The phenotype state pairs 1170, 1175 can then be processed by an indication engine 1180 to generate a predicted indication 1190 for the drug compound, as will be described in further detail in FIG. 12.

FIG. 12 illustrates an example of an indication engine, e.g., the indication engine 1180, constructing a distribution of predicted perturbed phenotype states and initial phenotype states and determining a discrepancy between the respective distributions to identify a predicted indication of a drug compound.

In particular, the indication engine 1180 can be configured to process the initial phenotype states 1170 and the perturbed phenotype states 1175 to generate corresponding distributions 1200, e.g., the initial phenotype state distribution 1230 and the perturbed phenotype state distribution 1220. The system can then analyze the distributions 1230 and 1220 to identify a discrepancy 1240 between the distributions indicative of a predicted indication with respect to a particular phenotypic indicator 1250, e.g., by determining that at least a first threshold number of phenotype state pairs have a measure of discrepancy greater than a second threshold for the phenotypic indicator. As an example, the system can use the count of the number of states 1260 to evaluate the phenotype state pairs with respect to the first threshold. In this case, the discrepancy 1240 can be evaluated with respect to a criterion for identifying a new indication, e.g., a particular level of change affected among a particular number of initial omics states, with respect to the particular phenotypic indicator 1250. In the particular example depicted, the engine 1180 is comparing the means of the distributions 1220, 1230 to determine the discrepancy 1240.

More specifically, the system can determine the discrepancy 1240 between each of the phenotype state pairs and can aggregate the discrepancy, e.g., using the number of omics states with a discrepancy greater than a threshold. As an example, the system can determine a measure of distance for each embedding of the initial phenotype state and each embedding of the perturbed phenotype state with respect to an embedding dimension corresponding with the phenotypic indicator 1250. As another example, the system can compare each embedding with an embedding of a target phenotype indicative of the phenotypic indicator 1250, e.g., using an inner product between the embeddings to calculate a similarity score. In this case, the system can determine the measure of similarity between the embedding of the initial phenotype state and the measure of similarity between the embedding of the predicted perturbed phenotype state and assess whether the discrepancy between the two measures is greater than a threshold number.

In the particular example depicted, the phenotypic indicator 1250 can be for weight, e.g., with the desired outcome of the repurposed drug being to affect weight loss in a subject. In this case, the system can analyze the distributions 1200, e.g., to assess if the mean weight of the initial phenotype state distribution 1220 decreased in a meaningful way, e.g., with respect to a criterion for identifying a new indication, in the perturbed phenotype state distribution 1230, e.g., as depicted in the graphs of the distributions 1200. As another example, the phenotypic indicator 1250 can be for LDL cholesterol, e.g., with the desired outcome of the repurposed drug being to affect a decrease in LDL cholesterol in a subject. In this case, the system can analyze the distributions 1200, e.g., to assess if the mean LDL of the initial phenotype state distribution 1220 decreased in accordance with the criterion for identifying a new indication in the perturbed phenotype state distribution 1230, e.g., as depicted in the graphs of the distributions 1200.

In the case that the indication engine 1180 determines that the discrepancy 1240 aligns with a criterion for identifying a new indication, the system can output a predicted indication for the particular drug compound. The predicted indication for the particular drug compound can then be used for downstream purposes, e.g., a user of the system can obtain the drug compound and perform physical experiments to test one or more properties of the drug compound, e.g., actual symptoms in subjects, with respect to the identified predicted indication to further evaluate the prediction. As another example, the system can determine that the drug compound should be administered to a subject for treating the predicted indication. In this case, the drug can be administered to a subject for treating the predicted indication.

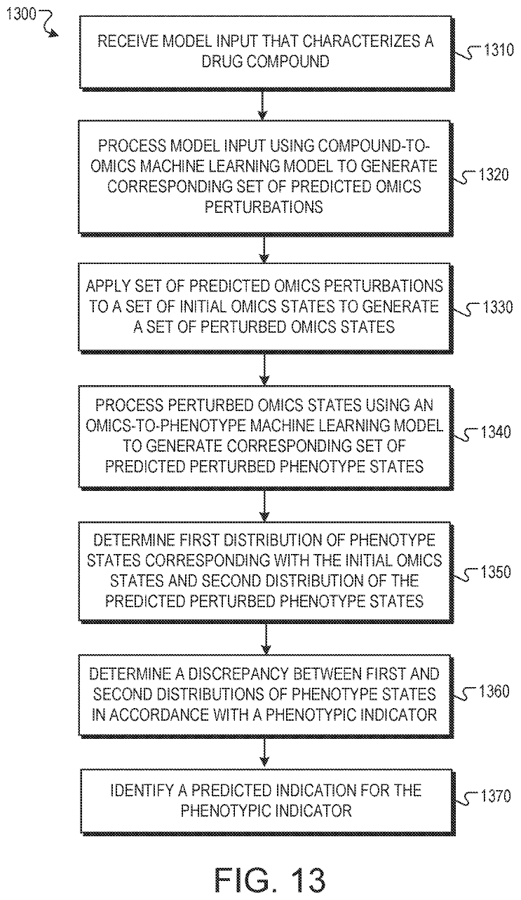

FIG. 13 is a flow diagram of an example process for predicting a new indication of a drug compound. For convenience, the process 1300 will be described as being performed by a system of one or more computers located in one or more locations. For example, an OPC unification system, e.g., the OPC unification system 100 of FIG. 1, appropriately programmed in accordance with this specification, can perform the process 1300.

In particular, the system can receive a model input that characterizes a drug compound (step 1310). As an example, the system can receive compound data that identifies a drug compound and an associated indication, e.g., a known target disease of the drug compound. In particular, the system can receive an embedding of a compound associated with a target gene that is known to be or predicted to be associated with a disease. In some cases, the system can process the compound data using the compound embedding model to generate a compound embedding as the model input.

The system can process the model input using the compound-to-omics machine learning model to generate a corresponding set of omics perturbations for the drug compound using the compound-to-omics machine learning model (step 1320). In particular, the system can use the compound-to-omics machine learning model to generate multiple omics perturbations for the drug compound. As an example, the compound-to-omics machine learning model can be configured to output a probability distribution of omics perturbations, e.g., a range of predictions instead of a single point estimate, e.g., by sampling from a posterior distribution generated using variational inference or Markov Chain Monte Carlo. As another example, the system can generate multiple omics perturbations using an ensemble of compound-to-omics machine learning models, e.g., an ensemble of models with different architectures or an ensemble of models that were trained with different initialization weights. As yet another example, the system can use dropout regularization to generate multiple omics perturbations by processing the model input multiple times with different dropout regularization weights.

In some cases, the compound-to-omics machine learning model can be further configured to output a likelihood of the predicted omics perturbation that can be used to determine the set of omics perturbations, e.g., by processing an intermediate output of the compound-to-omics machine learning model to output a corresponding likelihood of the predicted omics perturbation. The system can then use the likelihood to sample from the multiple omics perturbations. For example, the system can randomly sample from the omics perturbations or select the top N omics perturbations of the drug compound based on the predicted likelihood for each respective omics perturbation.

The system can then apply the set of omics perturbations to a set of initial omics states to generate a corresponding set of perturbed omics states (step 1330). In particular, the system can apply an embedding that characterizes the predicted omics perturbation to an embedding that characterizes the initial omics state to generate a corresponding embedding of the perturbed omics state, e.g., by combining, e.g., adding the embeddings. In some cases, the set of initial omics states can be selected from a set of possible omics states as a set of representative omics states. In other cases, the system can receive the set of initial omics states. In yet another case, the system can identify a set of phenotypes of interest and generate the set of initial omics states, e.g., using the phenotype-to-omics machine learning model.

The system can process the perturbed omics states using an omics-to-phenotype machine learning model to generate a corresponding set of predicted perturbed phenotype states (step 1340). In particular, the system can process each of the perturbed omics states to generate a corresponding predicted perturbed phenotype state. The system can then determine a first distribution of phenotype states corresponding with the initial omics states and a second distribution of the predicted perturbed phenotype states (step 1350). In some cases, the system can process the initial omics states to generate corresponding initial phenotype states, e.g., in order to determine the distribution of phenotype states corresponding with the initial omics states. In other cases, the system can have already received phenotype states of interest, e.g., to generate the initial omics states. In this case, the system can use the received phenotype states directly as the distribution of phenotype states corresponding with the initial omics states.

The system can determine a discrepancy between the first and second distributions of phenotype states in accordance with a phenotypic indicator (step 1360), and can identify a predicted indication for the phenotypic indicator (step 1370). In particular, the system can evaluate the first distribution, e.g., the initial phenotype distribution, and the second distribution, e.g., the predicted perturbed phenotype distribution, along an axis of variation for a certain phenotypic state of a subject. As an example, a phenotypic indicator can be weight, a level of cholesterol, blood pressure, or insulin, presentation of symptoms of a virus or infectious disease, etc. For example, the system can determine a measure of distance using an embedding of the initial phenotype state and an embedding of the predicted perturbed phenotype state, e.g., the system can subtract the embeddings with respect to a dimension corresponding with the phenotypic indicator or can determine a measure of similarity using an inner product between the respective embeddings and an embedding of a target phenotype for the phenotypic indicator.

More specifically, the system can determine a measure of discrepancy between each initial phenotype and associated predicted perturbed phenotype pair using the phenotypic indicator and can compare the measure of discrepancy to a criterion for identifying a new indication. In some cases, the system can determine that at least a particular number of phenotype state pairs have a measure of discrepancy greater than a threshold level of discrepancy for the phenotypic indicator, e.g., the system can determine: a (i) level of change between the initial phenotype state and the predicted perturbed phenotype state pair among (ii) a large enough number of phenotype state pair samples as meaningful evidence of a new indication for the drug compound, e.g., with respect to the criterion for identifying a new indication, e.g., a predicted indication for the drug compound.

In some cases, the predicted indication for the particular drug compound can then be used for downstream purposes, e.g., a user of the system can obtain the drug compound and perform physical experiments to test one or more properties of the drug compound, e.g., actual symptoms in subjects, with respect to the identified predicted indication to further evaluate the prediction. Furthermore, the system can determine that the drug compound should be administered to a subject for treating the predicted indication, e.g., the drug compound can be administered to the subject for treating the predicted indication.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non-transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially-generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry, e.g., an FPGA (field programmable gate array) or an ASIC (application-specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand-alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a data communication network.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read-only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer-readable media suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework, or a Jax framework.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method, the method comprising:
receiving data identifying a drug compound associated with a first indication;
processing a model input that characterizes the drug compound using a compound-to-omics machine learning model, wherein the compound-to-omics machine learning model is configured to process the model input to generate a predicted omics perturbation that would result from administering the drug compound to a subject, to generate a corresponding set of omics perturbations for the drug compound;
applying the set of omics perturbations to a set of initial omics states to generate a set of perturbed omics states;
processing the set of perturbed omics states using an omics-to-phenotype machine learning model, wherein the omics-to-phenotype machine learning model is configured to process a model input that characterizes an omics state to generate a predicted phenotype state corresponding to the omics state, to generate a corresponding set of predicted perturbed phenotype states for the drug compound;
determining a first distribution of phenotype states corresponding with the set of initial omics states and a second distribution of phenotype states using the predicted perturbed phenotype states; and
identifying a predicted indication for the compound using a determined discrepancy between the first and second distributions of phenotype states in accordance with a phenotypic indicator.

2. The method of claim 1, wherein receiving data identifying a compound associated with a first indication comprises receiving an embedding of a compound associated with a target gene that is predicted to be associated with a disease.

3. The method of claim 1, wherein processing the model input that characterizes the drug compound using a compound-to-omics machine learning model comprises processing an embedding of compound data.

4. The method of claim 1, further comprising determining a likelihood of the predicted omics perturbation, wherein determining the likelihood of the predicted omics perturbation comprises:
processing an intermediate output of the compound-to-omics machine learning model to output a corresponding likelihood of the predicted omics perturbation.

5. The method of claim 4, wherein the corresponding set of omics perturbations for the drug compound comprises a top N omics perturbations for the drug compound based at least on the predicted likelihood of the predicted omics perturbation.

6. The method of claim 4, wherein the corresponding set of omics perturbations for the drug compound comprises a random sample of omics perturbations for the drug compound from a subset of possible omics perturbations, wherein the subset comprises the omics perturbations with a corresponding likelihood greater than a threshold.

7. The method of claim 1, wherein the compound-to-omics machine learning model comprises an ensemble of compound-to-omics machine learning models that have been trained with one or more of different initializations or different architectures.

8. The method of claim 7, further comprising generating a plurality of omics perturbations using the ensemble of compound-to-omics machine learning models as the set of omics perturbations for the drug compound.

9. The method of claim 1, further comprising using dropout regularization to generate a plurality of omics perturbations as the set of omics perturbations by processing the drug compound a corresponding plurality of prediction iterations, wherein each prediction iteration is characterized by a first dropout rate.

10. The method of claim 1, further comprising:
receiving the set of initial omics states.

11. The method of claim 10, wherein receiving the set of initial omics states further comprises:
identifying a set of phenotypes of interest; and
processing the set of phenotypes of interest using a phenotype-to-omics machine learning model, wherein the phenotype-to-omics machine learning model is configured to process a model input that characterizes phenotype data to generate corresponding omics data, to generate the set of initial omics states.

12. The method of claim 1, wherein applying the set of omics perturbations to the set of initial omics states to generate a set of perturbed omics states further comprises, for each predicted omics perturbation and each initial omics state:
applying an embedding that characterizes the predicted omics perturbation to an embedding that characterizes the initial omics state; and
generating a corresponding perturbed omics state embeddings.

13. The method of claim 10, wherein processing the set of perturbed omics states for the drug compound further comprises processing a set of the perturbed omics state embeddings.

14. The method of claim 1, wherein determining a first distribution of phenotype states corresponding with the set of initial omics states comprises:
processing the set of initial omics state using the omics-to-phenotype machine learning model to generate the corresponding set of phenotype states.

15. The method of claim 1, wherein identifying a predicted indication for the compound using the determined discrepancy between the first and second distributions of phenotype states in accordance with the phenotypic indicator comprises:
determining a measure of discrepancy between each initial phenotype and predicted perturbed phenotype state pair in accordance with the phenotypic indicator, wherein each phenotype state pair comprises an initial phenotype state associated with a respective initial omics state and a predicted perturbed phenotype state associated with a respective perturbed omics state that represents applying a respective omics perturbation to the initial omics state; and
determining that at least a first threshold number of phenotype state pairs have a measure of discrepancy greater than a second threshold for the phenotypic indicator.

16. The method of claim 15, wherein determining the measure of the discrepancy between each initial phenotype and predicted perturbed phenotype state pair in accordance with the phenotypic indicator comprises:
determining a measure of distance between an embedding of the initial phenotype and an embedding of the perturbed phenotype state with respect to a dimension corresponding with the phenotypic indicator.

17. The method of claim 16, wherein determining a measure of discrepancy between each initial phenotype and predicted perturbed phenotype state pair in accordance with the phenotypic indicator comprises, for each phenotype state pair:
determining a first measure of similarity between an embedding of the initial phenotype state in the phenotype state pair and an embedding of a target phenotype indicative of the phenotypic indicator in accordance with an inner product of embeddings; and
determining a second measure of similarity between an embedding of the predicted perturbed phenotype state in the phenotype state pair and the embedding of the target phenotype indicative of the phenotypic indicator in accordance with an inner product of embeddings.

18. The method of claim 17, further comprising:
determining that that a discrepancy between the second measure and the first measure exceeds a first threshold number for a second number of phenotype state pairs.

19.-23. (canceled)

24. A system comprising:
one or more computers; and
one or more storage devices communicatively coupled to the one or more computers, wherein the one or more storage devices store instructions that, when executed by the one or more computers, cause the one or more computers to perform operations comprising:
receiving data identifying a drug compound associated with a first indication;
processing a model input that characterizes the drug compound using a compound-to-omics machine learning model, wherein the compound-to-omics machine learning model is configured to process the model input to generate a predicted omics perturbation that would result from administering the drug compound to a subject, to generate a corresponding set of omics perturbations for the drug compound;
applying the set of omics perturbations to a set of initial omics states to generate a set of perturbed omics states;
processing the set of perturbed omics states using an omics-to-phenotype machine learning model, wherein the omics-to-phenotype machine learning model is configured to process a model input that characterizes an omics state to generate a predicted phenotype state corresponding to the omics state, to generate a corresponding set of predicted perturbed phenotype states for the drug compound;
determining a first distribution of phenotype states corresponding with the set of initial omics states and a second distribution of phenotype states using the predicted perturbed phenotype states; and
identifying a predicted indication for the compound using a determined discrepancy between the first and second distributions of phenotype states in accordance with a phenotypic indicator.

25. One or more non-transitory computer storage media storing instructions that when executed by one or more computers cause the one or more computers to perform operations comprising:
receiving data identifying a drug compound associated with a first indication;
processing a model input that characterizes the drug compound using a compound-to-omics machine learning model, wherein the compound-to-omics machine learning model is configured to process the model input to generate a predicted omics perturbation that would result from administering the drug compound to a subject, to generate a corresponding set of omics perturbations for the drug compound;
applying the set of omics perturbations to a set of initial omics states to generate a set of perturbed omics states;
processing the set of perturbed omics states using an omics-to-phenotype machine learning model, wherein the omics-to-phenotype machine learning model is configured to process a model input that characterizes an omics state to generate a predicted phenotype state corresponding to the omics state, to generate a corresponding set of predicted perturbed phenotype states for the drug compound;
determining a first distribution of phenotype states corresponding with the set of initial omics states and a second distribution of phenotype states using the predicted perturbed phenotype states; and
identifying a predicted indication for the compound using a determined discrepancy between the first and second distributions of phenotype states in accordance with a phenotypic indicator.
